(19) United States
(12) Patent Application Publication
(10) Pub. No.: US 2025/0381334 A1
(43) Pub. Date: Dec. 18, 2025

(54) SYSTEMS AND METHODS FOR CUSTOMIZED PULSATILE PERFUSION CONTROL

(71) Applicant: THE BOARD OF REGENTS OF THE UNIVERSITY OF TEXAS SYSTEM, Austin, TX (US)

(72) Inventors: Juan M. Pascual, Dallas, TX (US); Muhammed Shariff, Dallas, TX (US); Obada Albaghdadi, Dallas, TX (US); Gabriel Reyes, Dallas, TX (US); Hadi Moussa, Dallas, TX (US); Mansur Syed, Dallas, TX (US); Jacob A. Awkal, Dallas, TX (US); Aksharkumar Dobariya, Dallas, TX (US)

(21) Appl. No.: 18/877,196

(22) PCT Filed: Jun. 23, 2023
(86) PCT No.: PCT/US23/69013

§ 371 Date: Dec. 19, 2024

Related U.S. Application Data
(60) Provisional application No. 63/354,978, filed on Jun. 23, 2022.
(60) Provisional application No. 63/488,385, filed on Mar. 3, 2023.

(51) Int. Cl. A61M 1/36; A01N 1/143; A61M 1/16; A61M 60/113; A61M 60/232; A61M 60/32; A61M 60/38; A61M 60/419; A61M 60/523; A61M 60/531; A61M 60/538; A61M 60/585

(52) U.S. Cl. CPC A61M 1/3666; A01N 1/143; A61M 1/1698; A61M 60/113; A61M 60/232; A61M 60/32; A61M 60/38; A61M 60/419; A61M 60/523; A61M 60/531; A61M 60/538; A61M 60/585; A61M 2205/502

(57) ABSTRACT

Aspects of the present disclosure generally relate to systems and methods for perfusion, and more specifically, for pulsatile blood perfusion based on a measured pressure waveform. One example method generally includes receiving, via a graphical user interface presented to a user, datapoints indicating a waveform; receiving one or more parameters associated with blood perfusion; generating an offset removed waveform based on the datapoints, the offset removed waveform having a physiological offset removed; converting the offset-removed waveform to a voltage waveform based on the one or more parameters; and operating, via the voltage waveform, a pump to provide blood in a perfusion system. The aspects described herein are applicable for any suitable perfusion environment, such as extracorporeal perfusion or isolated organ perfusion.

### CROSS REFERENCE TO RELATED APPLICATIONS

This application claims priority to U.S. Provisional Patent Application No. 63/354,978, filed Jun. 23, 2022 and titled “SYSTEMS AND METHODS FOR CUSTOMIZED PULSATILE PERFUSION CONTROL;” and U.S. Provisional Patent Application No. 63/488,385, filed Mar. 3, 2023 and titled SYSTEMS AND METHODS FOR CUSTOMIZED PULSATILE PERFUSION CONTROL;” the entireties of which are incorporated by reference herein.

### BACKGROUND

### 1. Technical Field

Aspects of the present disclosure relate to systems and methods for efficient extracorporeal perfusion control, and more specifically, perfusion control using a user friendly interface incorporating options for pulsatile blood perfusion based on measured physiological pressure or flow waveforms with enhanced applications in cardiopulmonary bypass and isolated organ perfusion.

### 2. Discussion of Related Art

Perfusion refers to the passage of fluid through the circulatory system to ensure delivery of vital nutrients and compounds to various concerted organ systems in order to preserve life. From oxygen delivery to its involvement in homeostatic maintenance of metabolite and physiological parameters, adequate perfusion is the key to protecting a patient from the onset of both acute and chronic pathophysiological conditions. Extracorporeal control of perfusion may be implemented using various electrical components.

### SUMMARY

Certain aspects of a blood perfusion method. The method generally includes receiving, via a graphical user interface presented to a user, datapoints indicating a waveform; receiving one or more parameters associated with blood perfusion; generating an offset removed waveform based on the datapoints, the offset removed waveform having a physiological offset removed; converting the offset-removed waveform to a voltage waveform based on the one or more parameters; and operating, via the voltage waveform, a pump to provide blood in a perfusion system.

Certain aspects of the present disclosure are directed towards a method for blood perfusion. The method generally includes receiving, via a graphical user interface presented to a user, datapoints indicating a pressure or flow waveform, receiving one or more parameters associated with the blood perfusion, generating a pressure or flow waveform by eliminating an offset to the native pressure or flow waveform acquired, converting to voltage, amplifying the waveform, and reintroducing corresponding voltage offset in order to convert the pressure or flow waveform to a communicable voltage waveform based on the one or more parameters inputted, and operating, via the voltage waveform, a centrifugal pump to provide perfuse blood.

Certain aspects of the present disclosure are directed towards a system for blood perfusion. The system generally includes a memory, and one or more processors coupled with the memory, the one or more processors configured to: receive, via a graphical user interface presented to a user, datapoints indicating a pressure or flow waveform; receive one or more parameters associated with the blood perfusion; generate a pressure or flow waveform by inputting a native pressure or flow waveform, which can optionally include the diastolic offset remove; converting the offset waveform to a voltage waveform based on the user input of systolic pressure, flow, or voltage parameter, a diastolic pressure, flow, or voltage parameter, and Beats Per Minute (BPM) through gain magnification of voltage waveform based on user input and followed by corresponding diastolic voltage offset reintroduction to generate appropriate range of voltage points to be outputted to a programmable mechanical pump; and operate, via the voltage waveform, said pump to provide blood in a perfusion system in a specific pulsatile manner or continuous flow depending on mode selected.

Certain aspects of the present disclosure are directed towards a non-transitory computer-readable medium having instruction stored thereon, which when executed by one or more processors, causes the one or more processors to: receive, via a graphical user interface presented to a user, datapoints indicating a pressure waveform, flow waveform, motor RPM feedback waveform; receive one or more parameters associated with the blood perfusion; generate a pressure or flow waveform by inputting a physiological native pressure or flow waveform with offset optionally removed; leading to voltage conversion and magnification following offset reintroduction programmatically to the inputted waveform based on user input of desired systolic pressure, flow, or voltage, diastolic pressure, flow, or voltage, and BPM parameters; and operate, via the voltage waveform, a mechanical pump to provide blood in a pulsatile or continuous manner depending on mode selected to the perfusion circuit.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates a two-dimensional schematic of a heart, in accordance with certain aspects of the present disclosure.

FIG. 2 is a graph illustrating pressure and volume waveforms of a human heart per cardiac cycle.

FIG. 3 illustrates a perfusion system, in accordance with certain aspects of the present disclosure.

FIG. 4 illustrates physiological waveform readings of taken from the aortic root.

FIG. 5 illustrates a native Aorta physiological waveform, in accordance with certain aspects of the present disclosure.

FIG. 6 illustrates a native Aorta physiological waveform with physiological offset removed, in accordance with certain aspects of the present disclosure

FIG. 7 illustrates a physiological waveform response to a voltage input waveform, in accordance with certain aspects of the present disclosure.

FIG. 8 illustrates a perfusion system, in accordance with certain aspects of the present disclosure.

FIG. 9 illustrates operation of a perfusion system, in accordance with certain aspects of the present disclosure.

FIG. 10 is a data flow diagram describing the operation of perfusion system, in accordance with certain aspects of the present disclosure.

FIG. 11A and FIG. 11B illustrate a graphical user interface (GUI) for receiving a pressure waveform and presenting vitals, in accordance with certain aspects of the present disclosure.

FIG. 12 is a flow diagram illustrating example operations for blood perfusion, in accordance with certain aspects of the present disclosure.

FIG. 13 illustrates an example computing device, in accordance with certain aspects of the present disclosure.

FIGS. 14A-14C illustrate an example operation of a perfusion system including an extracorporeal pulsatile circulatory control (EPCC), in accordance with certain aspects of the present disclosure.

FIGS. 15A and 15B illustrate an example angiography from using the EPCC, in accordance with certain aspects of the present disclosure.

FIG. 16 illustrates an example system for performing an aortic isolation procedure, in accordance with certain aspects of the present disclosure.

FIG. 17 illustrates an example system for performing a brachiocephalic artery isolation procedure, in accordance with certain aspects of the present disclosure.

FIGS. 18A and 18B illustrate an example measurements of cerebral activity under EPCC, in accordance with certain aspects of the present disclosure.

FIGS. 19A-19C illustrate example bar graphs indicating oxygenation, native cerebral pressure, and temperature values maintained by an EPCC system, in accordance with certain aspects of the present disclosure.

FIGS. 20A-20H illustrate example microscopic images of a brain cortex following a EPCC procedure, in accordance with certain aspects of the present disclosure.

It will be apparent to one skilled in the art after review of the entirety disclosed that the steps illustrated in the figures listed above may be performed in other than the recited order, and that one or more steps illustrated in these figures may be optional.

### DETAILED DESCRIPTION

Impaired perfusion has been documented to be either directly or indirectly involved in a countless array of conditions ranging from cardiac arrest and strokes to organ necrosis as a result of vessel calcifications, thrombotic embolisms, or hypovolemic shocks to name a few. Treatments for each are often surgically invasive in nature. Patients with cardiac arrests, valve stenosis, or congenital cardiac malformations are often subjected to extended cardiopulmonary bypass while surgeons perform surgery on the heart. The standard approach to cardiopulmonary bypass involves the utilization of extracorporeal perfusion equipment incorporating at least a pump for positive pressure control and a suitable lung bypass oxygenator. These two components are important to ensure perfusion is maintained while vital physiological processes can still be carried out. While the use of roller pumps has been the primary means of pressure generation in an extracorporeal perfusion circuit for decades, the incorporation of a centrifugal pump has shown significant merit and has been proven to increase surgical operation time while on cardiopulmonary bypass given its reduced hemolysis, a significant factor to ensure blood viability. Unlike predecessors which generate primitive sinusoidal pulsatile flow through the squeezing of tubing against the roller blade, introducing significant shear, the centrifugal pump utilizes continuous flow (nonpulsatile). While the necessity of pulsatile flow has been subject to debate in academia, several alarming neurovascular and metabolic morbidities have been documented under continuous flow when compared to pulsatile, leading to a genuine need for extracorporeal perfusion advancement. The present disclosure addresses the need for pulsatile flow while incorporating the benefits of utilizing a centrifugal pump by controlling a centrifugal pump to output a personalized irregular pulsatile pressure or flow waveform consistent with native measured waveform recordings taken directly from a specific patient or subject.

Current perfusion technology setups involve the direct evaluation and manual control of multiple discrete apparatuses to ensure adequate perfusion and that physiological levels are within established parameters within a patient. The shifting between multiple control mechanisms and graphical user interfaces to evaluate sensor readouts is not only taxing for perfusionists but also increases the probability of error, impacting lives. While a perfusionist may be modulating the flow rates to increase blood pressure, the perfusionist is in need of multitasking to ensure the other considerations are being met physiologically by shifting between multiple apparatuses. The present disclosure addresses this concern as well by eliminating the need to control and evaluate multiple isolated external components individually. Each component is interfaced with a single controller for optional software based control, manual control, sensor readouts, and feedback operations in some cases. The perfusionist may be responsible for making adjustments while evaluating key data all by using one graphical user interface, enhancing efficiency.

In the past decade, closed loop isolated organ perfusion has seen significant developments that has contributed to the increased longevity and viability of transplant organs. These systems, which attempt to maintain physiological functionality during transit between donor and recipient often incorporate pulsatile flow, pressure, oxygenation, and maintain standard metabolic homeostasis. As opposed to the current standard of “ice bath” preservation during transit, this mechanism has been shown to increase the lifespan of organs multifold. Nevertheless, the present disclosure may serve as an isolated organ preservation control unit. With the utilization of a single interface communicating with a controller for generalized component interfacing, the controller-component system can serve to establish an isolated perfusion loop pending interfacing with size reduced modular components. The applications of which will allow for a miniaturized cardiopulmonary bypass with additional functionality for drug/metabolite infusion associated with a single transplant organ. The modularity of the conception may allow for translocation into multiple applications in addition to those listed above.

Certain aspects of the present disclosure provide methods and systems for blood perfusion based on a pressure or flow waveform input. For example, datapoints may be obtained that define an irregular waveform, which may correspond to an aortic/brachiocephalic pressure or flow measurement.

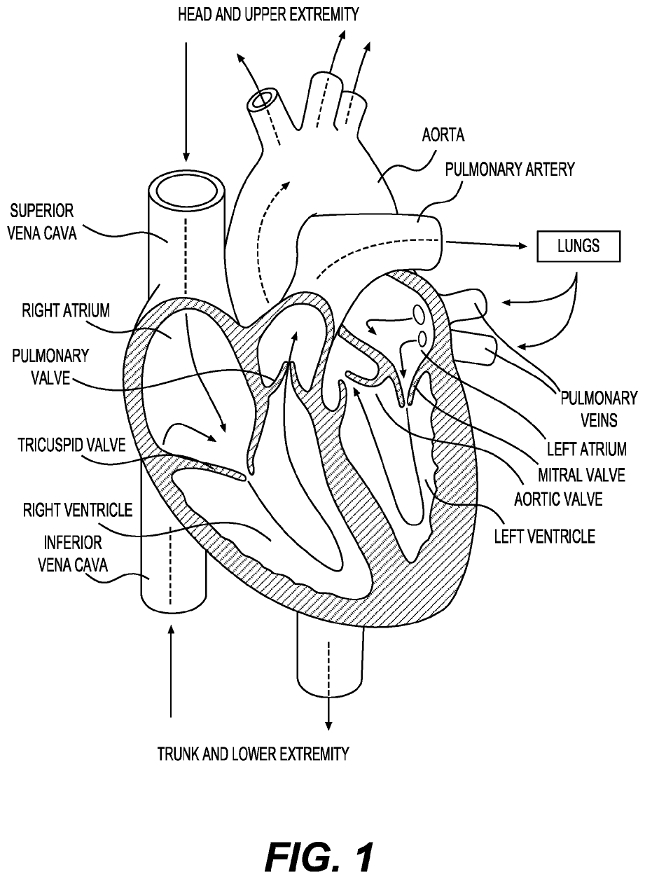

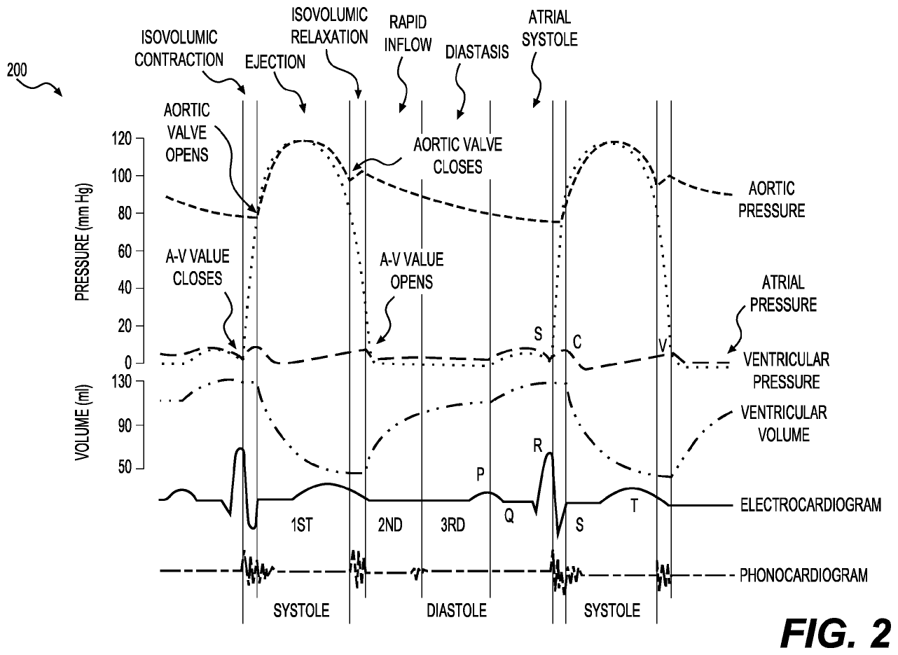

FIG. 1 illustrates a two-dimensional schematic of a heart, in accordance with certain aspects of the present disclosure. Blood is transmitted to organs in a pulsatile waveform which is consistent throughout the rest of the body. As the pulsation is propagated from the aorta (shown in FIG. 1) to the extremities, physiological pressure waveform attenuation in coordination with arterial contractility differences lead to variations in perfusion pressure and flow waveforms at different distal arterial organ sites. Considering this dilemma, it is important to mimic native physiological pulsatile waveforms characterized at native sites in an isolated organ perfusion system or mimic native pulsatile waveforms taken directly from a patient's aortic root for incorporation of the system in cardiopulmonary bypass. Characteristic physiological pressure and flow established during a cardiac output cycle taken directly at the aorta are shown in FIG. 2.

FIG. 2 is a graph 200 illustrating pressure and volume waveforms of a human heart per cardiac cycle. Although pulsatile waveform generation is of concern to ensure validity of the system, various other physiological parameters need to be controlled within range parameters to establish homeostasis. Therefore, there is a need to simultaneously control physiological parameters in conjunction with perfusion. Of these parameters, some of the most vital are pO2, pCO2, temperature, flow rates, pH, hemolysis, and metabolite and electrolyte concentration homeostasis. pO2 refers to pressure of dissolved oxygen in the blood used to calculate oxygen concentration. pCO2 refers to pressure of carbon dioxide in the blood used to calculate carbon dioxide concentration. pH is a scale used to measure the acidity or basicity of a solution. Hemolysis is indicative of Red Blood Cell death and may result in blood clot formation, preventing perfusion access to vital organs. To achieve homeostasis, the system may be expected to control such parameters as well as establish negative feedback mechanisms via sensor readouts. By employing strategies to tackle each of these physiological needs, the longevity and viability of the protocol and implementation can be extended

In some cases, for analysis, a porcine model may be used. Widely available, porcine subjects serve as a gateway to more human centered approaches given their similarity in cardiovascular anatomy, neurovascular function, cardiac functionality, and metabolism responses. Nevertheless, the pulsatile nature of porcine waveforms is similar but not the same as those found in humans. Therefore, it is important to retrieve these waveforms from porcine subjects for regional cardiac analysis. Simulation of the waveform retrieved directly from the aortic root or point of cannulation may be used to ensure the system accurately mimics physiological function. Therefore, it is important to dynamically control a programmable mechanical pump to mimic cardiac function. The mechanical pump head design may have well defined shear rates that will mitigate hemolysis and preserve the red blood cells.

Current methodologies available on the market with similar aims include Left Ventricular Assist Devices (LVAD) which allow for positive pressure allocation from the left ventricle to the aorta in an attempt to ensure that adequate blood pressure is maintained. Current LVADs are portable and can allow a patient to maintain normal routines. Unfortunately, LVADs involve the use of a patient's lungs for oxygenation and, therefore, strictly serve as positive pressure pumps. The use of an LVAD is of value for the purposes of establishing desired pressure differentials for short term use. The lack of native specific pulsatile flow in conjunction with concerning rates of hemolysis caused by shear induced by a rotary or centrifugal pump lead to many LVAD failures and result in coagulation of the perfusate. Despite its positive pressure capabilities, shear introduction in conjunction with the requirement of native lung usage and niche applications make this a nonviable option for extended cardiopulmonary bypass and isolated organ perfusion. Other non-portable alternatives are available as well, such as Extracorporeal Membrane Oxygenator (ECMO) and standard heart-lung bypass machines. Both are similar in nature. Perfusate is extracted from the venous return and sent to an oxygenator and heat exchanger to re-establish normal oxygen saturation and PO2 levels as well as maintain normothermic blood temperature. To bypass the function of the heart, these systems include a rotary pump which establish standard sinusoidal waveforms of differing amplitude and frequencies depending on the desired settings. The use of a rotary pump against the surface of medical grade tubing leads to the introduction of shear, thereby making the perfusate more susceptible to hemolysis. In addition, the standard sinusoidal waveform output is uncharacteristic of a regional specific pressure waveform seen in vivo. The utilization of a centrifugal pump, while limiting hemolysis, operates based on non-pulsatile flow, which has been documented to cause neurovascular and metabolic morbidities in patients. Therefore, there is a need for a system that is capable of specific waveform output at various frequencies and pressures as well as allow for the careful control of hemolysis to allow for extended intervals of usage.

It is possible to directly control a gear pump through the use of an established waveform input using a standard workbench software and controller. Nevertheless, the gear pump utilized may not be clinically approved and may be subject to significant FDA testing to ensure safety and prevention of extensive hemolysis (e.g., used in industrial mechanical engineering applications). Other devices have various disadvantages, such as lack of interfaceability with other components and affecting of waveforms by downstream load. RPM control of a centrifugal pump may be used, in some implementations. For example, a centrifugal pump may be used to establish pulsatile perfusion through the constructive interference mechanism, which establishes the summation of sinusoidal waves at varying frequencies phased out from one another to mimic physiological waveforms. The modulation of the frequencies of these two sinusoidal waves may be used to create a waveform categorically similar to a hepatic portal artery waveform. Nevertheless, to create multiple forms of waveforms may involve multiple sinusoidal waves to achieve with significant analysis to determine correct phase. This can be done using a Fourier analysis and phase diagram evaluation.

The present disclosure incorporates unique characteristics, including the amplification of waveforms to increase pressure or flow ranges as well as BPM potential all the while controlling standard clinically approved magnetically coupled centrifugal pump heads to generate physiologically relevant pulsatile pressure or flow waveforms. In addition, this approach uses the properties of the extracted waveform taken directly from a patient/subject to establish the waveform characteristics desired for user friendly integration and operation. Lastly, unlike many other mechanisms, the approach provided herein interfaces components with one controller which is in direct communication with a single GUI for control, feedback, and data evaluation all through one interface. This enhances efficiency and limits errors that may develop. However, the aspects of the present disclosure are not to be limited to usage of a single controller or a single GUI, and may be implemented using multiple controllers or GUIs, in some implementations.

In certain aspects, with the use of controllers, actuators, and sensors, specific pulsatile perfusion is achieved using sensitive and adaptive technologies with suitable response times and settling times. Certain aspects provide for measurement of the blood flow profile at a circulatory site of interest with accuracy. The isolated live brain pulsatile perfusion system described herein allows the use of collected waveforms and dynamically controls a shear-resistive centrifugal pump to output irregular waveforms corresponding to the arterial systolic and diastolic values in addition to modulating the heart rate. Furthermore, incorporating a graphical user interface (GUI) in coordination with a controller specific software platform may be used to retrieve physiological and mechanical data while establishing feedback to maintain physiological parameters within specified ranges. This will help achieve perfusion using region specific cardiac waveforms from physiologically relevant ranges of, for example, 20 to 140 mmHg and 40 to 180 BPM. Values between these parameter ranges are commonly seen in vivo. Nevertheless, this is not the limit of the system's capabilities. Values approaching the extreme ends of these parameter ranges may be associated with cardiac function compromise. In addition, subject focused physiological vitals (pO2, temperature, pH, electrolyte concentration, etc.) as well as pressure and blood flow readings are documented by interfaceable blood parameter analyzers, pressure catheters, in-line and perivascular flow probes respectively. Oxygen levels are controlled using perfusion oxygenators used in surgery. Temperature is regulated using an external convective heater. Data is saved for post experimentation data analysis. Given the desire for experimental protocol manipulation for metabolic analysis as well as evaluation of pharmacokinetic/pharmacodynamic drug profiles, the system is capable of encapsulating the effects of variable introduction via a rate-controlled infusion pump system.

It has been established that region specific pulsatile pressure waveform generation as demonstrated by pressure catheter readouts taken directly from the aortic root of porcine subjects is close to identical to the input waveform established depending on the input established.

While some examples provided herein describe a perfusion system for isolated brain perfusion to facilitate understanding, the system may be used for isolated perfusion for any suitable organ. The overall method system perfusion can be subdivided into two steps: perfusion device system integration and software-hardware interfacing. A prerequisite to establishing an physiologically relevant bypass can be to directly cannulate both arterial tubing from the system to the subject directly at the aortic root, a brachiocephalic artery, or at a site of arterial entry for an isolated organ, while the venous cannula should be placed at the point of entry into the right atrium (Superior vena cava), or a venous return from a selected organ. Integration of the perfusion device will occur during surgery.

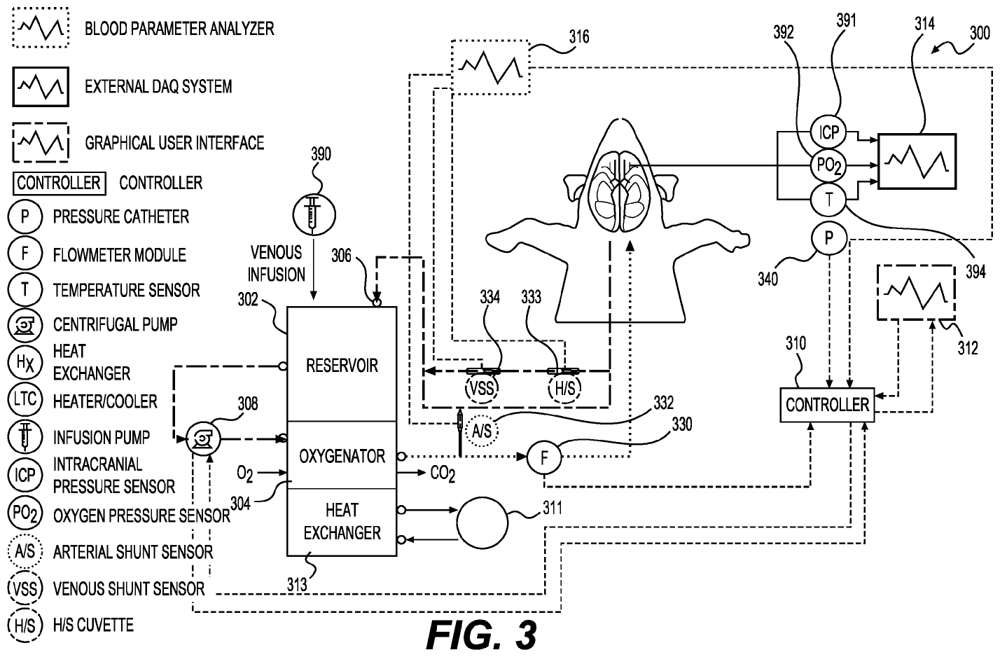

FIG. 3 illustrates a perfusion system 300, in accordance with certain aspects of the present disclosure. The order listed below is not subjected to set order classifications and components may be moved around the circuit if deemed fit from an application perspective To prime the perfusion system prior to the incorporation of blood perfusate, a reservoir 302 may be filled with Normosol or an equivalent solution. A shear resistive magnetic centrifugal pump head may be incorporated to create positive pressure to establish flow from the reservoir to the oxygenator 304 and then to the rest of the biological circuit. During the priming phase, the initial closed reservoir-oxygenator system is primed to provide proper fluid-solid interfacing with the tubing and to reduce air bubbles that could potentially form. Following priming of the circuit and incorporation of the perfusate, cardiopulmonary bypass may occur. The pressure catheter and/or flow probe, which may also be used to take physiological readings, may be placed in the aortic root and/or connection point of cannula for isolated organ perfusion to be used in feedback response and for systolic and diastolic pressure and/or flow evaluation. The circuit pathways may be understood by establishing reservoir 302 as the initial point of reference. After incorporating the perfusate into the venous return component 306 of the reservoir, the blood is filtered to remove large insoluble particles and degassed to remove air bubbles that could develop during reservoir decantation. The filtered blood is sent to an adjacent compartment to serve as the filtered blood to be released to the pump 308. The pump 308 may be a centrifugal pump drive with a magnetically coupled pump head to establish high rotations per minute (RPM). Magnetic coupling of a shear-resistant pump head to the pump drive may impact hemolysis outcomes. Varying the RPM leads to pressure differentials that will allow for the introduction of pulsatile positive pressure or flow. This pump drives the flow of the perfusate towards the subject or isolated organ and characteristically allows for return flow from the extremities (Superior vena cava or a corresponding return vein). In some aspects, the pump head outlet may be connected to the input of the heater oxygenator component located underneath the reservoir. The heater component 311 is connected directly with an external heat exchanger 313 which continuously supplies metal coils of the heat exchanger with water with an established temperature. For example, water from metal coils of the heat exchanger flow to the heater component 311, warmed, and returned back to the heat exchanger. The blood is warmed by the oxygenator heat exchanger through a convective means and maintains the temperature established on the heat exchanger fluid interface. Following the blood passing through the heat exchanger, the blood is then passed into the oxygenator column through which the blood is oxygenated to partial pressure levels established via a manual oxygen pressure mixer with medical air. This allows for oxygenated blood to be controlled to levels desired. The output of the oxygen mixer is directly sent through an isoflurane-controlled chamber with a percentage modulator connected in series with the tubing if an anesthesiologist deems this a suitable means for anesthesia control. Depending on the concentration percentage, the output of the pressure mixer may pick up isoflurane on the way to the oxygenator. If anesthesia control is not desired through the use of the system, the concentration modulator can be bypassed allowing only desired oxygen to be sent. The final bit of tubing is sent through an air filter before connecting to the lower connection point on the oxygenator. The perfusate, upon interaction with the oxygenator, adjusts partial pressures of oxygen and isoflurane (if deemed necessary) and is then ready to be sent into circulation.

From the circuit directly, the arterial cuvette, following calibration using a blood gas analyzer calibrator, can be connected to the blood parameter analyzer. Blood, following oxygenation, will flow out of the oxygenator towards the aorta of the subject. A tiny shunt will allow for limited blood flow to be shunted from the main arterial line towards the cuvette 333. The shunt uses low blood velocities to gather data. The perfusate will return via a connection to the main venous return or reservoir directly. The main line tubing used may be a medical-grade tubing. The same mechanism may be established for venous return as well. Another shunt may be established to read venous return blood values as well. Hematocrit saturation values are read via a venous shunt sensor 334 placed in series with the main line tubing while the venous sensor connected in parallel with the main line tubing follow a configuration similar to the arterial sensor. Perfusate from both the arterial and venous shunt will flow back into the main venous line or by direct connection to the reservoir following analysis. As shown, the measurements by the cuvette 333, and sensors 332, 334 may be provided to a blood parameter analyzer 316 for analysis, which may be then provided to a controller 310 for connection to the GUI for display of blood parameter values graphically and/or used to trigger feedback control through the incorporation of the infusion pump if deemed useful.

Following oxygenation, the main line tubing may be connected in series with an ultrasonic extracorporeal flow probe to evaluate circuit flow readings via Doppler analysis. The tubing output from the flow probe connects directly with the arterial cannula in the subject. Clamping at the regions of an interface may be performed during cannulation. To avoid air bubble formation, it may be of benefit to pour saline into both connections for the tubing and tap the tubing. Using the same method for arterial cannulation, the venous tubing can be connected to the cannula placed at the vena cava, thereby completing the circuit. To promote proper flow and functioning of the newly established circuit, the pump may be allowed to run on continuous flow mode for a few minutes at the desired flow/pressure inputted.

Finally, metabolite analysis and control are capable via an infusion pump. The metabolite in focus may be placed in a syringe and deposited via controlled infusion using an interfaceable syringe infusion pump 390. Foreign sensors evaluating uncommon metabolites or drugs can be directly incorporated via shunts, placed in line with the tubing, or directly placed into the reservoir. Upon command and/or feedback, the infusion pump is expected to start and stop the infusion of the metabolite of interest.

As shown, the perfusion system 300 may include a controller 310 which may generate a signal to control the pump 308. For example, the controller 310 may be coupled to a display providing a graphical user interface 312 that facilitates the presentation of data to users and reception of control parameters such as a pressure or flow waveform to be used to control blood flow via the pump 308. As shown, various sensors may be used to measure the parameters of the perfusion system, which may be provided to controller 310 to control blood flow or pressure. For example, a pressure sensor 340 (e.g., pressure catheter) may be used to measure a pressure associated with the blood flow through vessels and provide an indication of the pressure to controller 310. The controller 310 may consider the pressure measurement when controlling the pump 308. The perfusion system 300 may also include a flowmeter module 330, which may measure the blood flow and provide a flow measurement to the controller 310. As shown, there may be feedback measurement values from the pump 308 to the controller 310, allowing the closed-loop system to control the pump 308 accurately with voltage waveform adjustments based on the feedback. As shown, other measurements via sensors such as an oxygen pressure sensor 392, intracranial pressure sensor 391, and temperature sensor 394 may be provided to a data acquisition system (DAQ) 314 for storage and display.

Certain aspects of the present disclosure are directed toward techniques for establishing selective pulsatile flow as well as user control and evaluation of multiple parameters by perfusionist which may, for example, be all through a single interface in some implementations. For example, a waveform generation component may be used to establish pulsatile pressure waveforms with accuracy to physiological readings. Given the introduction of a pressure catheter during the initial stages of surgery, the pressure waveform of the subject may be acquired. A pressure waveform can be isolated and used to control the centrifugal pump head (e.g., pump 308) to mimic physiological pressure outputs.

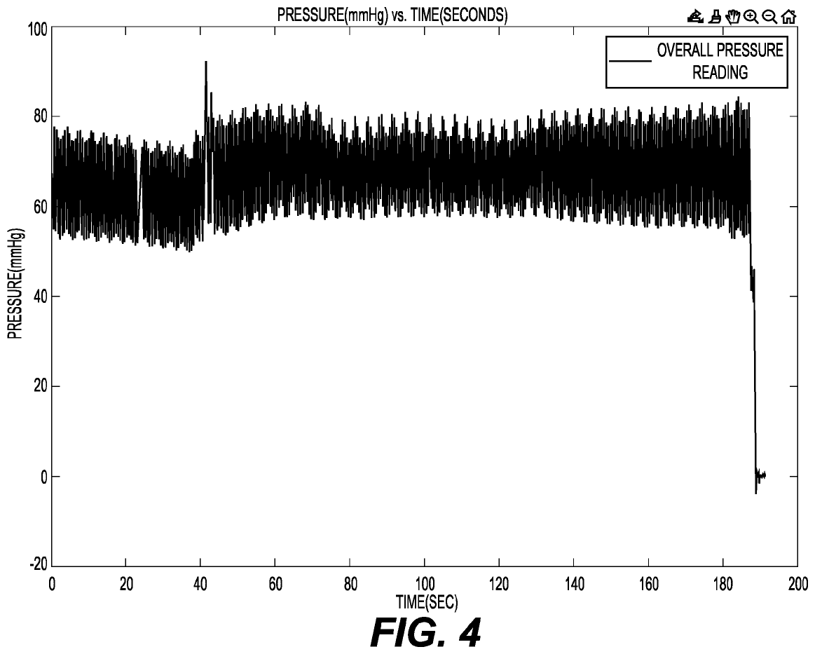

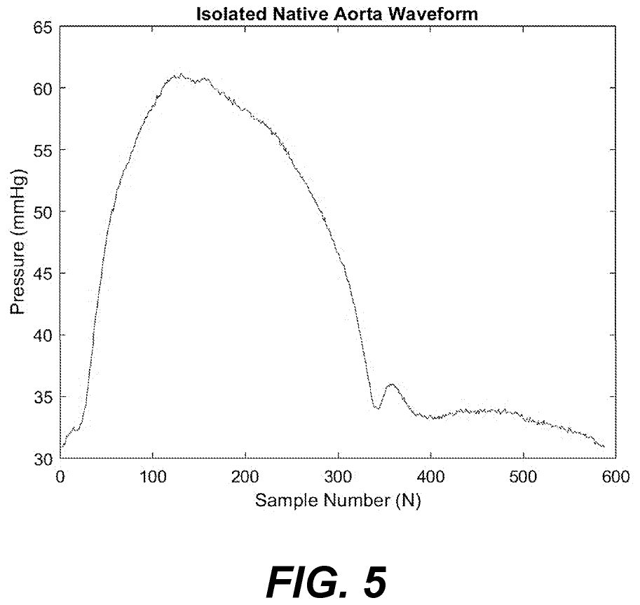

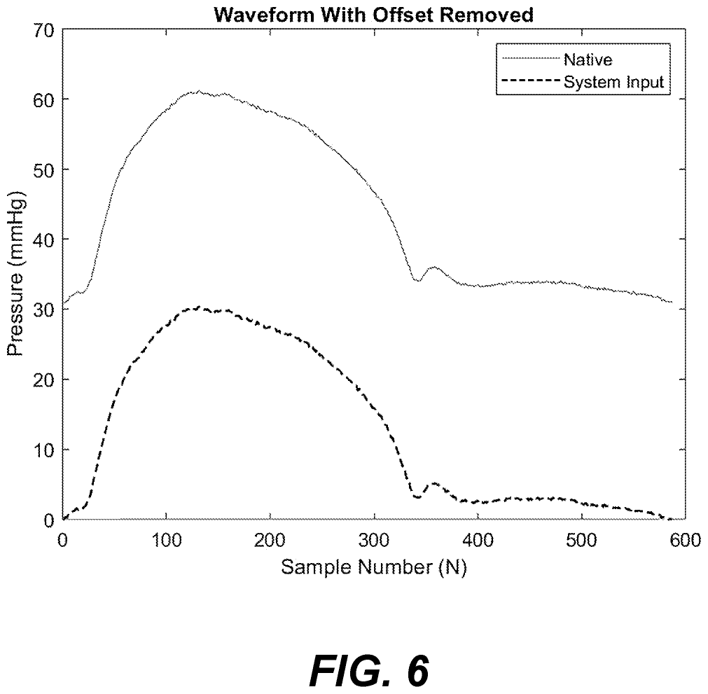

FIG. 4 illustrates physiological waveform readings taken from the aortic root of a pig subject. Using the pressure or flow waveform reading, one representative waveform may be isolated. Depending on the sampling rate and overall data points, a specific time range may be selected and a data samples corresponding to a single native waveform may be isolated. Following range deliberation, sampled beats may be filtered and processed to be used for specific pulsatile perfusion. For a specific selected beat, within a time range, The recreated values may be identical considering the heart capabilities in some scenarios. The value differentials when selecting a native waveform may be small and are usually less than 0.1-0.2 mmHg off one another. It may be important to isolate a waveform with equal minimum values to correspond to a modifiable waveform. To accomplish such a task, waveform points may be isolated during the systolic rise due to its overall linear nature or by isolation via determining the two diastolic values of a waveform through evaluating the minimum after sending the signal through a lowpass filter. Once a representative waveform is isolated as shown in FIG. 5, the diastolic pressure or flow is determined using a minimum function in a third party software (e.g. Excel or Matlab). Each data point can be subjected to a subtraction by this diastolic pressure/flow to remove any offset prior to saving the waveform as a .CSV. Once the offset is removed as shown in FIG. 6, the waveform can be uploaded to the program for conversion into a communicable voltage waveform. Generally, in accordance with Nyquist sampling rates, the smaller the sampling rate will allow for the better response of the pump.

FIG. 5 illustrates an aorta pressure waveform, in accordance with certain aspects of the present disclosure. Following signal processing, the waveform isolation is shown in FIG. 5. Data for this waveform isolation may be taken directly from the aortic root of the subject prior to initiation of cardiopulmonary bypass of the subject. Once the waveform has been isolated, the waveform may be further processed to control the pump. Depending on the actual pulse pressure or flow value and desired systolic and diastolic pressure or flow value, each data point is converted to a corresponding voltage value to represent a gain magnification associated with appropriate scaling followed by the reintroduction of a voltage offset corresponding to desired diastolic pressure/flow. The heart rate is modulated by the control of time delay between the output of each data point in the waveform.

The user can input pressure or flow, or voltage readings on a GUI corresponding to the minimum and maximum pressures or flows. The difference between the desired systolic and diastolic pressures is determined and is then used to serve as a factor for gain amplification of the inputted waveform. Each data point within the data set for the zeroed isolated waveform can then be amplified to a corresponding voltage, pressure, or flow desired using this factor and desired inputs and offset by diastolic pressure, flow, or voltage serving as the offset. The pressure or flow readings are converted to interfaceable voltage readings for the pump 308 and is sent via an analog signal to the pump 308 directly which modulates the RPM of the pump head to correspond to the voltage input.

In some aspects, the overall frequency of the beat may be modified. The inputted waveform (e.g., as shown in FIG. 6) may be used to account for one beat. The duration may be set as 1 second for the inputted waveform. Therefore, for example, if on the GUI, a user inputs 60 BPM or one beat per second, the number of data points within the data set may be outputted every second. Therefore, if there are, for example, 400 data points within the inputted data file representing the waveform, within one second, 400 data points will be outputted, or 1 data point will be outputted every 1/400 seconds. To modulate frequency, this threshold may be modified. If the user indicates 120 BPM, the 400 data points may be outputted every half a second. Therefore, a general relationship of:

$$\frac{1}{f \times datapoints}$$

may be used to determine the wait time for the controller to output data points in which f corresponds to frequency in Hz and datapoints corresponds to data points of the file indicated the pressure waveform as provided via the GUI. The data points of the file may be automatically determined by determining the array size of the file. The GUI, therefore, may only receive user input for frequency during this process, in some aspects.

The controller 310, which is controlled by a communicable GUI 312, is capable of both controlling the system and receiving feedback, allowing to serve as the main interface to both control the system as well as evaluate vital readout. The user selects pressure, flow, or voltage outputs for systolic along with desired diastolic pressure, flow, or voltage outputs and frequencies. The user may also switch between manual and automatic control of the pump via a GUI software-hardware switch. The user may also save pressure readings from the pressure catheter and various sensors of the system for further data analysis. The user may also input desired data sampling rates for experimentation purposes. Based on the inputted data sampling rates, the sampling rate associated with one or more sensors as described herein may be controlled. The user may input desired file size, and the system will automatically create a new file with same file name and incrementation (e.g. filename1 . . . filename2 . . . filenameX) once the original file size is filled. This feature allows for automatic control of data acquisition without user intervention. The user may, on the GUI, visualize in real-time the pressure readings and waveforms, flow readings and waveforms, voltage input readings and waveforms, pump functionality waveforms for RPM, and desired sensor readouts along with blood parameter readings, as described. These visualizations may change dynamically depending on the user's input and may be displayed on various graphs.

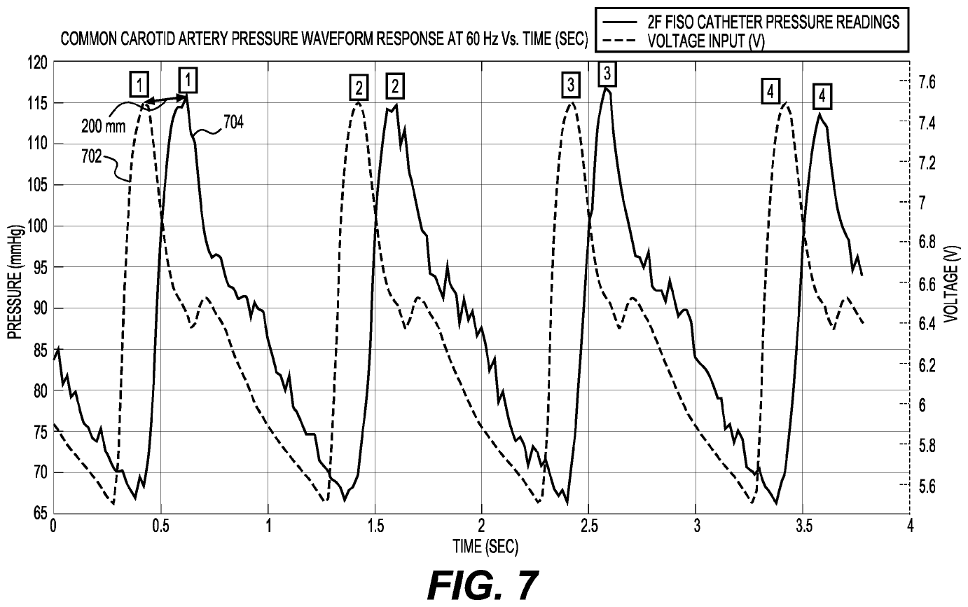

For isolated organ perfusion, FIG. 7 illustrates a blood pressure response to a voltage waveform, in accordance with certain aspects of the present disclosure. FIG. 7 provides a sample of the system efficacy. The waveform 702 corresponds to the inputted voltage waveform established at voltage readings of 7.5V for systolic and 5.5V for diastolic at a frequency of 60 BPM. The waveform 704 corresponds to the output of the system in response to an input of a brachial artery waveform for testing, however other organs may be utilized during preservation. These inputted values corresponded to a 118 mmHg systolic and 65 mmHg diastolic, showing waveform accuracy. The data is sampled at 50 samples/second. The system's output is in synch with the input of the pump.

The aspects described can provide blood perfusion using a commercial pump head with a standard linear flow shear rate value of less than 1500 dynes/cm². The perfusion system is compatible with blood perfusate and/or other non-Newtonian fluid and maintains sterility. The system may be integrated into an isolated organ model. The system allows input of digitized specific clinical pressure waveforms mimicking irregular waveforms characteristics of native cardiac function. The system is capable of exogenous metabolite, drug, and/or perfusate input into the system. The system also provides a rate of blood flow through an isolated model that meets and exceeds physiological ranges. The net internal system operating conditions are compliant at physiological temperature ranges and exceed above and below normothermic temperatures. The pulsatile flow numerical waveform generator in the system may be implemented with a touch screen based graphical user interface (GUI) with physiologically relevant input ranges of 20 to 140 mmHg for both systolic and diastolic pressure and 40 to 180 BPM pulsatile waveform input frequencies.

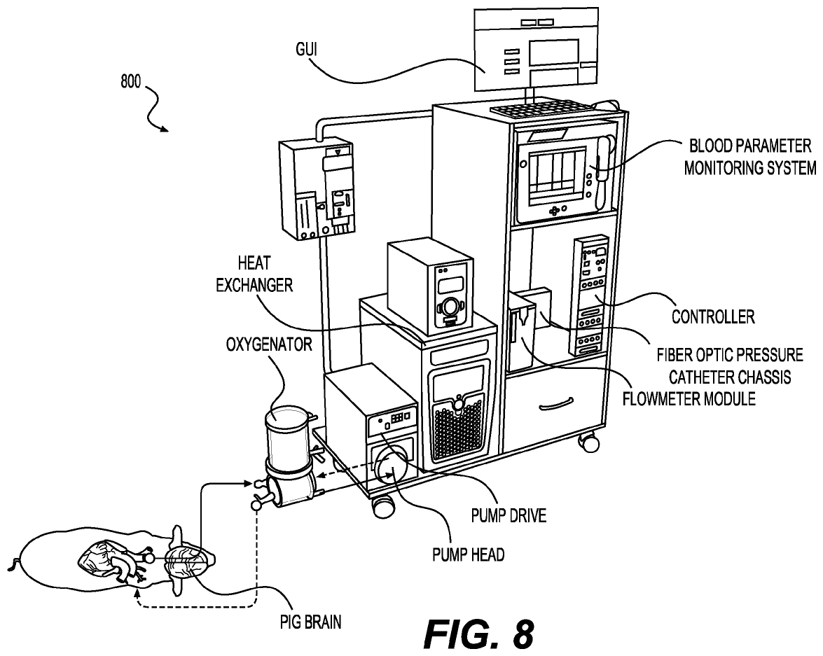

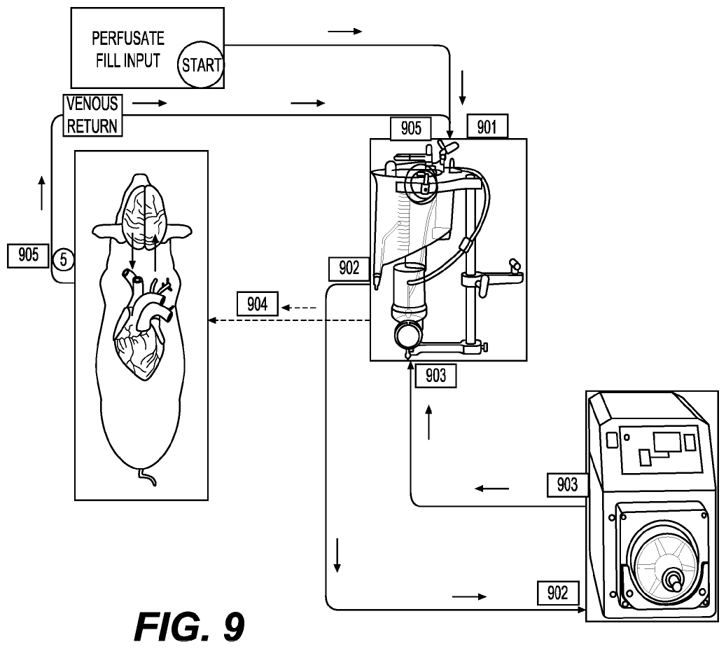

FIG. 8 illustrates a perfusion system 800, in accordance with certain aspects of the present disclosure. As shown, a host computer is connected to a touch screen monitor, on which the GUI is provided. The host computer is also connected to a controller, which allows for the host computer to receive data from the various sensors as well as communicate with the pump. The controller may have various modules, each module allowing for connection with either outputs or inputs of varying types. As shown, the perfusion system 800 can also include a blood parameter monitoring system (e.g., corresponding to analyzer 316 of FIG. 3). As shown, the controller may control a pump drive that is driving a pump head. As shown, the pump head is coupled to an oxygenator and heat exchanger, as described herein. The various components and their operation are described in more detail with respect to FIG. 9.

FIG. 9 illustrates operation of a perfusion system, in accordance with certain aspects of the present disclosure, however, the order of components can be modified depending on what the perfusionist deems fit and based on application specificity. The perfusate of choice is first introduced via the reservoir, entering through a venous entrance at 901. The perfusate is then sent through a filter within the reservoir and then to the pump head at 902, which pumps according to the voltage output to the pump head, as described herein. The perfusate is then sent from the pump head to the heat exchanger at 903, where the temperature is regulated according to a set temperature, which heats the perfusate by convection. The perfusate makes its way up the heat exchanger and into the oxygenator where oxygenation and gas transfer occur. Oxygen tanks are connected directly to the input line of the oxygenator port. To modulate oxygen levels, the manual pressure control on the oxygen tank can be varied. The flow then goes directly from the oxygenator out to the porcine model at 904, where the surgeon can cannulate the aorta or artery of interest. After perfusing the porcine model, the return cannula from the venous return allows return perfusate to travel back into the oxygenation circuit at 905.

The perfusion system also includes a monitoring system (e.g., corresponding to analyzer 316) for monitoring of a multitude of parameters including pH, pO2, pCO2, temperature, oxygen saturation, etc. These parameters allow for continuous monitoring of essential parameters to preserve the subject's life during perfusion. The monitoring system uses proprietary cuvettes to analyze blood parameters. These are established as shunts off both the arterial and venous lines to reduce flow velocity for maximal accuracy in analysis, as described herein. If deemed necessary, a hematocrit saturation sensor can be placed in line with the main tubing and interfaced with the blood parameter analyzer. In some aspects, sensor information can be sent continuously every 6 seconds to the host via RS-232 communication protocols for display, data acquisition and saving, and/or feedback. Rearrangement of sensor attachments may be implemented depending on the particular experimental application.

The heat exchanger, which as described, connects directly to the heater/oxygenator. The controls of the heat exchanger allow for the user to manually input the desired temperature to the nearest 0.1° C. The heat exchanger may be connected via an inlet and outlet tube that provides a water jacket for the perfusate which runs continuously and is heated externally.

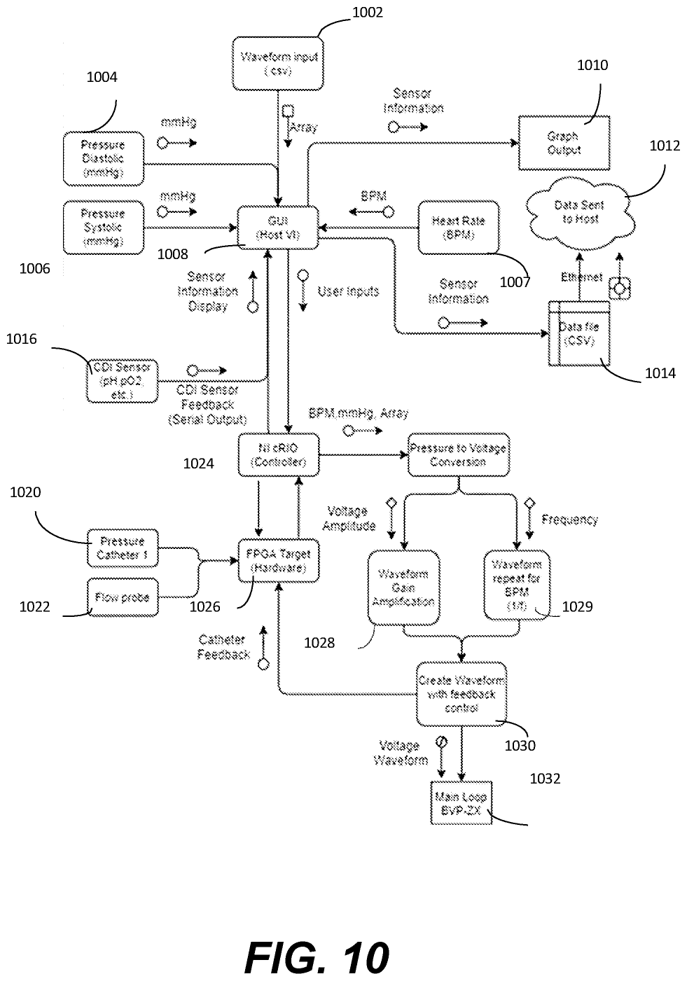

FIG. 10 is a data flow diagram describing the operation of perfusion system, in accordance with certain aspects of the present disclosure. As shown, the inputs the user provides include both systolic and diastolic pressures, flows, or voltages 1004, 1006, heart rate (BPM) 1007, as well as a pressure or flow waveform input 1002. These values are input via the GUI 1008 (e.g., corresponding to GUI 312), as described, and are sent to the controller 1024 (e.g., corresponding to controller 310). The controller 1024 then sends the data through multiple conversions and calculations to eventually output a voltage to the pump. Data 1016 is also received from the pressure catheter or flow probe and the monitoring system (e.g., for monitoring multitude of parameters including pH, pO2, pCO2, temperature, oxygen saturation, etc.) and are displayed graphically (e.g., graph 1010) and saved as data files on the host computer based on user desired data sampling rates. The controller provides the BPM, pressure, flow, or voltage inputs, and waveform to a pressure or flow to voltage conversion component of the software 1018, which may convert the pressure or flow waveform to a voltage waveform based on pulse pressure or flow (e.g., by as described herein). At block 1028, a waveform gain amplification may be applied to the voltage amplitude of the voltage waveform followed by a reintroduction of an offset voltage corresponding to desired diastolic pressure/flow, and at block 1029, a waveform may be repeated for setting the BPM may be implemented, to create the waveform at block 1030. The frequency associated with the voltage waveform may be set based on equation:

$$\frac{60}{BPM \times ( number of samples )}$$

The voltage gain applied at software block 1028 may be set in accordance with equation:

$$\frac{Systolic V - Diastolic V}{Systolic mmHg - Diastolic mmHg} mmHg + Diastolic V$$

Where Systolic V is the systolic voltage input from the user (manual) or voltage determined following pressure or flow calibration conversion given user input (Feedback), the diastolic V is the diastolic voltage input from the user (manual) or voltage determined following pressure or flow calibration conversion given user input (Feedback), systolic mmHg is the systolic pressure input from the user, and the diastolic mmHg is the diastolic voltage input from the user.

The waveform may be provided to the main loop 1032 (e.g., the pump), as shown. In some cases, the generated waveform at block 1030 may be fed back for comparison with pressure and flow measurements at respective blocks 1020, 1022. The hardware block 1026 (e.g., implemented using a field-programmable gate array (FPGA)) may compare the pressure and flow measurements with the generated waveform and provide feedback to controller 1024 to adjust the input to the pressure to voltage converter (e.g., conversion component 1018) accordingly if feedback is employed.

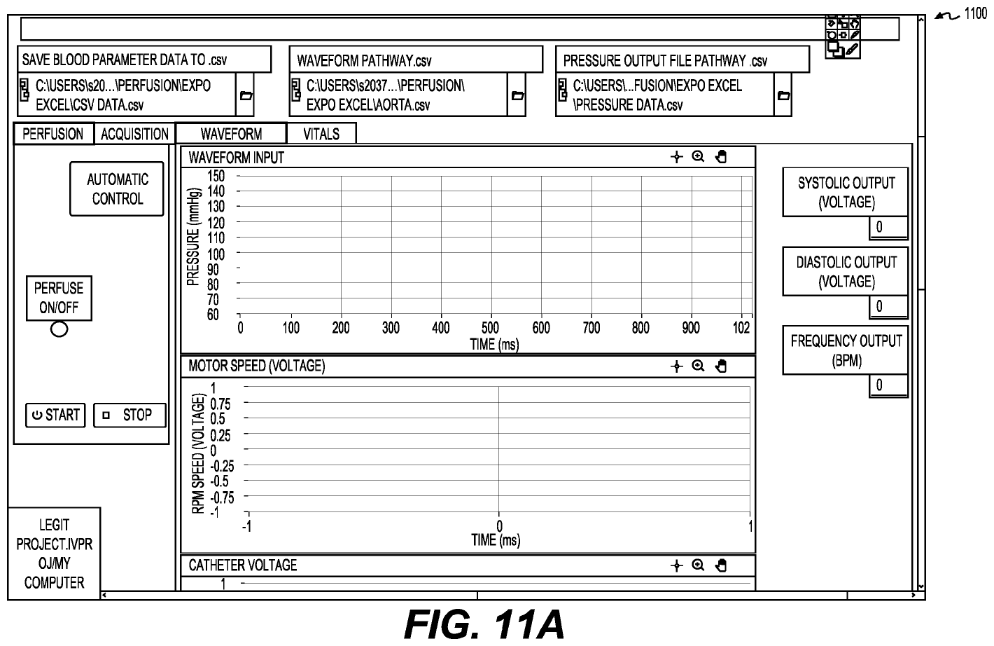

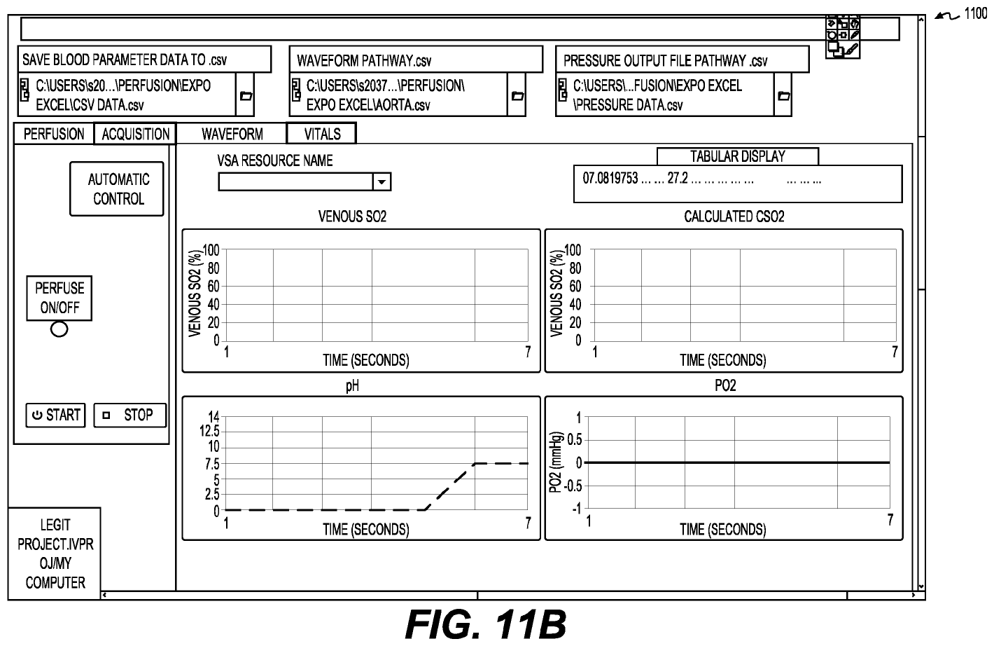

FIG. 11A and FIG. 11B illustrate a GUI 1100 for receiving a pressure and flow waveforms and presenting vitals, in accordance with certain aspects of the present disclosure. As shown, the GUI allows a user to save blood parameter data that may be sensed by various sensors as described herein. The use may also input a pressure or flow waveform file. GUI may also allow the user to save data read from the pressure catheter, flow probe, voltage input to pump, corresponding and voltage RPM output. Once the pressure or flow waveform is provided, the user may begin perfusion, resulting in the conversion of the pressure or flow waveform to a voltage waveform for operating a pump, as described herein. The user also has the option to maintain continuous flow by either switching to manual control by click on the button on the interface and adjusting the RPM on the pump directly, or increasing diastolic voltage, pressure, or flow to match systolic values. There may be tabs that account for perfusion and acquisition controls. Under the acquisition tab, a user may select the appropriate sampling frequency desired for data sampling.

The user may be prompted via a dialog box to begin inspection of the system to ensure all connections are appropriately established. Upon inspection, the user may be prompted to input systolic pressure (mmHg), flow, or voltage, diastolic pressure (mmHg), flow, or voltage, and frequency (BPM). The threshold range for each may correspond to between 20 and 300 for pressure and 20 and 200 for frequency, in some cases. The systolic pressure must be greater than or equal to the diastolic. The equality of the two variables establishes continuous flow at the established pressure output or if manual control setting is pressed, the pump RPM setting can establish continuous flow as well. Upon satisfaction of these parameters, a proceed button may become active and visible. Upon clicking, perfusion is ready, which then leads to the user turning on and running the RT controller. Operation is characterized by a dynamic waveform output graphs for pressure (waveform and catheter), and voltage (input pump voltage and RPM). If the user decides to change the parameters, the user may do so at any time by changing the number in the numerical control and pressing proceed. In the meantime, perfusion at previous parameters continues. If the user would like to pause the experiment, the user may do so by selecting pause, once perfusion begins and vice versa for resume. Unlike the stop button, pause will pause the program from acquiring data and will require a user to hit manual to establish manual control In addition, the data acquisition will be paused until resume is pressed. On the front panel, the “perfusion ON/OFF” indicator is established to determine if perfusion is taking place. If the indicator is green and the text says “Automatic mode is ON’, perfusion is currently underway. The pressing of this button can be the last step to proceed to software-controlled perfusion. With this button not pressed, “Perfusion ON/OFF” can be deactivated. If the indicator is grey or white and/or the text says ‘Manual control is ON’, perfusion control from the system has stopped. The “Manual Control is ON” button may be depicted by default. If turned off, signified by the button turning navy blue, this indicates that the control for the pump is being established manually via pressing the buttons on the pump directly for continuous flow control. This is useful for the perfusionists to prime the oxygenator circuit without the need to adjust parameters on the software and establish pulsatile perfusion. To access visual representation of vitals, the user has the option to select the vitals tab as demonstrated in the FIG. 11B. Under the vitals tab, a graphical representation of the transient changes in pH, pO2, sO2 are established to demonstrate impact on the parameters by experimental procedures over time. Additional vital parameter graphs can be incorporated easily upon desired selection if user deems it necessary.

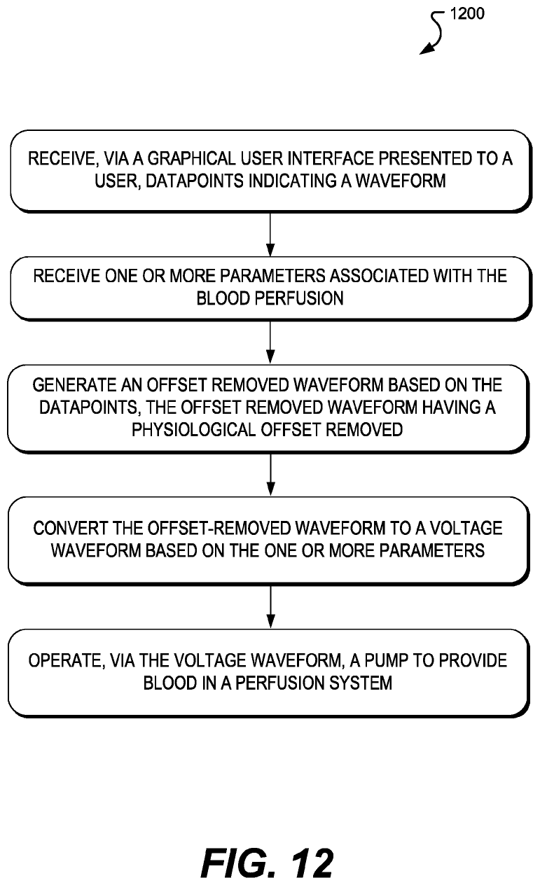

FIG. 12 is a flow diagram illustrating example operations 1200 for blood perfusion, in accordance with certain aspects of the present disclosure. The operations 1200 may be performed by, for example, a controller such as the controller 310 or computing device 1300.

The operations 1200 begin, at block 1202, with the controller receiving, via a graphical user interface (e.g., GUI 1100) presented to a user, datapoints indicating a pressure or flow waveform. The pressure or flow waveform may include an irregular waveform. The pressure waveform may include a portion of an aorta or brachiocephalic waveform measurement.

At block 1204, the controller receives one or more parameters associated with the blood perfusion. The one or more parameters may include at least one of a BPM parameter, a systolic pressure, flow, or voltage parameter, or a diastolic pressure, flow, or voltage parameter. Receiving parameters may include receiving, systolic, diastolic, BPM via the graphical user interface. During perfusion, any parameter may be modified while other parameters may remain the same. The datapoints may be outputted (e.g., at a particular rate) for conversion to the voltage waveform based on the BPM parameter.

At block 1206, the controller generates a waveform based on a waveform, the waveform having a physiological offset removed. For example, the controller may receive a pressure or flow waveform which can also include the diastolic offset removed, such that each value in the array is subtracted by the diastolic value. Therefore, the smallest value of the waveform with the offset removed is 0 (corresponding to offset), while the maximum value is systolic minus diastolic. This waveform is fed into the system. The system then amplifies the offset removed signal accordingly based on the gain established by the systolic and diastolic inputs followed by an addition of the diastolic offset at the end to establish the appropriate systolic and diastolic voltage values to be sent to the pump. At block 1208, the controller converts the offset pressure or flow waveform to a voltage waveform based on the one or more parameters.

At block 1210, the controller operates, via the voltage waveform, a pump (e.g., centrifugal pump) to provide blood in a perfusion system. For example, a rotation per minute (RPM) of the pump may be varied based on the voltage waveform. The pump may include a magnetically coupled pump head, in some aspects.

In some aspects, receiving the one or more parameters may include receiving, via the graphical user interface presented to a user, at least one of a systolic pressure parameter, a diastolic pressure parameter, a systolic pressure voltage, or a diastolic pressure voltage. The controller may amplify the voltage waveform based on the at least one of the systolic pressure parameter, the diastolic pressure parameter, the systolic pressure voltage, or the diastolic pressure voltage. Operating the pump may include providing the amplified voltage waveform to the pump.

In some aspects, the controller may receive, from one or more sensors, at least one of a pressure measurement or a flow measurement from the perfusion system, the method further comprising generating the pressure waveform based on the at least one of the Native pressure measurement or the flow measurement.

In some aspects, the perfusion system may include a reservoir (e.g., reservoir 302), and an oxygen pressure mixer. The pump may be coupled between the reservoir and the oxygen pressure mixer and configured to provide a blood flow from the reservoir to the oxygen pressure mixer. The perfusion system may also include a heat exchanger (e.g., heat exchanger 313) configured to warm up the blood to be provided to the oxygenator. In some aspect, blood from the oxygen pressure mixer is provided, via an isoflurane-controlled chamber, to the oxygenator. In some aspects, blood from the oxygenator flows to an aorta of a subject. In some aspects, the perfusion system further comprises a shunt path coupled between a sensor and an arterial line coupled to an output of the oxygenator. In some aspects, an input of the reservoir is coupled to a venous line, the perfusion system further comprising a shunt path from the venous line to a sensor.

The present disclosure provides techniques centered around the concept of enhancing cardiopulmonary bypass approaches in addition to isolated organ perfusion, and is not to be limited to isolated brain perfusion. Additionally, currently for perfusionists, each system component is often isolated from one another and each requires its own attention from the perfusionist to ensure successful cardiopulmonary bypass. For the system provided herein, each of these isolated components are interfaced under one controller system for integrated evaluation, allowing for feedback control based on physiological signals, to control from one single interface. Swapping out compatible pump technologies (e.g., with lower cardiac output potential) will allow for the same mechanisms to be allocated to a close circuit with just one organ, enhancing lifetimes for organ transplants.

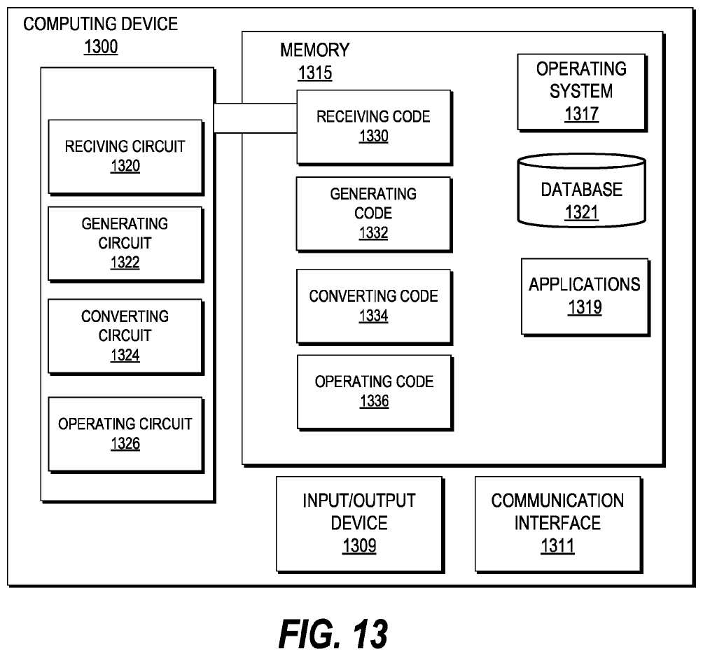

FIG. 13 illustrates an example computing device 1300, in accordance with certain aspects of the present disclosure. The computing device 1300 can include a processor 1303 for controlling overall operation of the computing device 1300 and its associated components, including input/output device 1309, communication interface 1311, and/or memory 1315. A data bus can interconnect processor(s) 1303, memory 1315, I/O device 1309, and/or communication interface 1311.

Input/output (I/O) device 1309 can include a microphone, keypad, touch screen, and/or stylus through which a user of the computing device 1300 can provide input and can also include one or more of a speaker for providing audio output and a video display device for providing textual, audiovisual, and/or graphical output. Software can be stored within memory 1315 to provide instructions to processor 1303 allowing computing device 1300 to perform various actions. For example, memory 1315 can store software used by the computing device 1300, such as an operating system 1317, application programs 1319, and/or an associated internal database 1321. The various hardware memory units in memory 1315 can include volatile and nonvolatile, removable, and non-removable media implemented in any method or technology for storage of information such as computer-readable instructions, data structures, program modules or other data. Memory 1315 can include one or more physical persistent memory devices and/or one or more non-persistent memory devices. Memory 1315 can include, but is not limited to, random access memory (RAM), read only memory (ROM), electronically erasable programmable read only memory (EEPROM), flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by processor 1303.

Communication interface 1311 can include one or more transceivers, digital signal processors, and/or additional circuitry and software for communicating via any network, wired or wireless, using any protocol as described herein. Processor 1303 can include a single central processing unit (CPU), which can be a single-core or multi-core processor (e.g., dual-core, quad-core, etc.), or can include multiple CPUs. Processor(s) 1303 and associated components can allow the computing device 1300 to execute a series of computer-readable instructions to perform some or all of the processes described herein. Although not shown in FIG. 13, various elements within memory 1315 or other components in computing device 1300, can include one or more caches, for example, CPU caches used by the processor 1303, page caches used by the operating system 1317, disk caches of a hard drive, and/or database caches used to cache content from database 1321. For implementations including a CPU cache, the CPU cache can be used by one or more processors 1303 to reduce memory latency and access time. A processor 1303 can retrieve data from or write data to the CPU cache rather than reading/writing to memory 1315, which can improve the speed of these operations. In some examples, a database cache can be created in which certain data from a database 1321 is cached in a separate smaller database in a memory separate from the database, such as in RAM or on a separate computing device. For instance, in a multi-tiered application, a database cache on an application server can reduce data retrieval and data manipulation time by not needing to communicate over a network with a back-end database server. These types of caches and others can be included in various implementations and can provide potential advantages in certain implementations of software deployment systems, such as faster response times and less dependence on network conditions when transmitting and receiving data.

Referring to FIG. 13, the processor 1303 and/or memory 1315 may be used to implement geometric contouring. For example, processor 1303 may include circuit 1320 for receiving, circuit 1322 for generating, circuit 1324 for converting, and circuit 1326 for operating.

The memory 1315 may be coupled to processor 1303 and may store code which, when executed by processor 1303, performs the operations described herein. For example, the memory 1315 may include code 1330 for receiving, code 1332 for generating, code 1334 for converting, and code 1336 for operating.

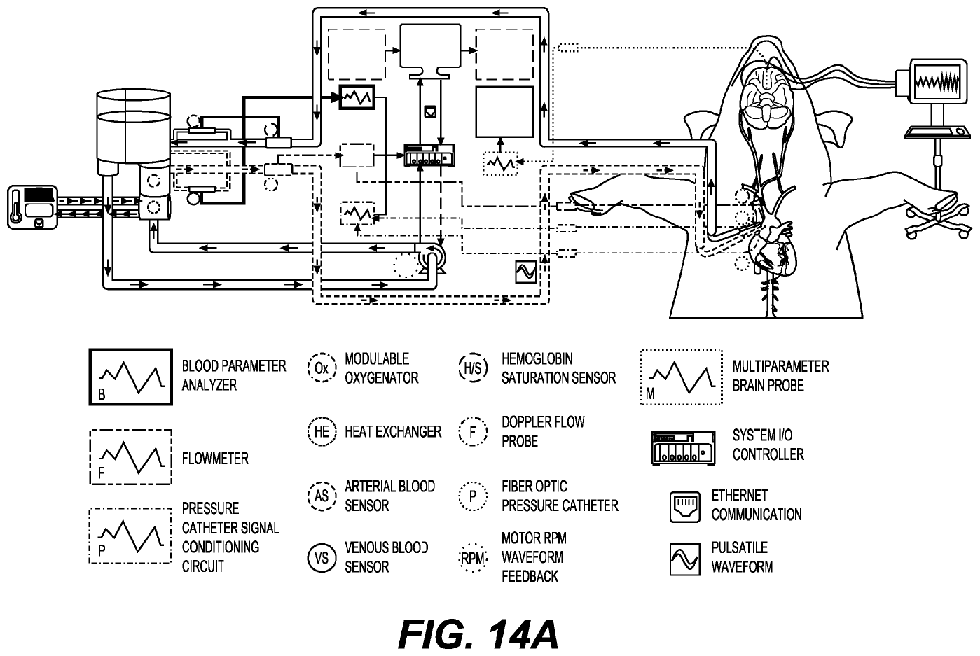

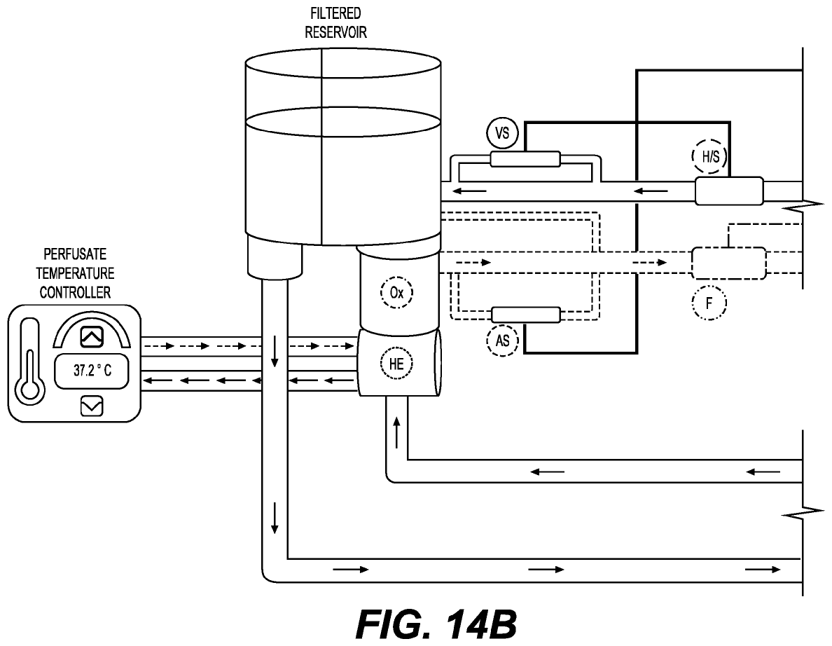

FIGS. 14-20H depict an example use case scenario of an extracorporeal pulsatile circulatory control (EPCC) system being used with a pig.

For instance, FIGS. 14A-14C illustrates a full EPCC system. Custom-developed software can provide multiparameter physiological data visualization in a single interface and in real time via connection to a compactRIO (e.g., National Instruments).

In some examples, selective vascular access to the brain is beneficial in metabolic tracer, pharmacological and other studies aimed to characterize neural properties independently of somatic influences from chest, abdomen or limbs. However, artificial control of cerebral circulation can interfere with pulsatility-dependent vascular signaling or abolish high-order neural network phenomena such as the electrocorticogram, even when individual neuronal activity is preserved. Thus, the techniques disclosed herein render cerebral hemodynamics fully regulable by mechanical means to replicate or modify native pig brain perfusion. To this end, vascular separation of the head from the systemic circulation via the aorta or brachiocephalic artery can be followed by transition to full extracorporeal pulsatile circulatory control (EPCC). This control relied on a computerized algorithm that maintained, for several hours, blood pressure, flow and pulsatility at near-native values individually measured just before EPCC. Continuous electrocorticography and brain depth electrode recordings can be used to evaluate brain activity relative to the standard offered by awake human electrocorticography. Under EPCC, this activity remained generally unperturbed compared to the native circulation state, as did cerebral oxygenation, pressure, temperature and microscopic structure. Thus, our approach provides the study of neural activity and its circulatory manipulation in independence of most of the rest of the organism.

Cerebral blood flow manipulation can facilitate numerous experimental contexts such as the investigation of brain function under ischemia and/or stroke or the study of the action and distribution of pharmacological agents or metabolic tracers. In some of these studies, selective vascular access to head or brain is desirable to characterize neural properties as independently as possible from somatic influences stemming from unavoidable native or experimentally induced changes in the rest of the organism. This is because metabolism or multi-organ distribution of biologically active substances can present insurmountable limitations to accurate kinetic analyses. Among these influences, blood flow and pressure alterations and extracerebral metabolism can impose significant and often nonlinear effects on brain function. To circumvent these limitations, the techniques disclosed include vascular disconnection followed by artificial provision of the mechanical, cellular and chemical circulatory support and control that the rest of the body exerts on the brain. A subsequent objective can provide, via this regulation, the future investigation of a broad variety of physiological and pathological circulatory states.

To this end, a computer-controlled mechanical perfusion and blood composition regulatory system (e.g., as depicted in FIGS. 14A-14C) can provide direct coupling to the arterial and venous circulation of the pig. The system can be referred to as an extracorporeal pulsatile circulatory control (EPCC) system. The EPCC system can operate guided by five physiological considerations or parameters.

First, the system can use pulsatile flow or linear flow. The system depicted in FIGS. 14A-14C depict pulsatile flow instead of linear flow. Pulsatility can provide improvements for maintaining human extracerebral organ function. The reasons can stem from animal and cellular observations. On the one hand, pulsatility impacts, at least in experimental animal pathophysiological states, regional and global cerebral blood flow (CBF), cerebral metabolic rate of oxygen (CMRO2), cerebral delivery of oxygen (CDO2) and cerebral vascular resistance (CVR), and this may exert neural function repercussions since the recovery from global brain ischemia can be influenced by pulsatility. On the other, and despite some uncertainties about specific cell signaling mechanisms, cerebral endothelial cells, pericytes and astrocytes are baroreceptive and exhibit heartbeat frequency-range responsiveness. This phenomenon is associated with the regulation of molecules relevant to neurovascular coupling and other facets of neural control. Thus, pulsatility can minimize or avoid disruption of potentially significant biological mechanisms.

Second, given the normal variability of circulatory function across pigs, which is also noted in man and other animal research models, and the potential for further increase of this variability under experimental interventions, individualized measurements prior to EPCC in each pig can be performed. This can customize the EPCC to maintain circulation to the brain as close as possible to the native state of each animal following a negligible delay upon vascular diversion.

Third, broad modulatory capacity of blood pressure, pulsatility, flow and as many cellular and chemical composition parameters as desirable can make EPCC useful for a variety of potential experimental situations

Fourth, although blood substitutes are available, each pig's autologous blood can be retained and supplemented with heterologous whole blood. This can minimize chemical composition changes while maintaining red blood cell and brain metabolic relations.

Fifth, higher order neural activity can be persevered as reported by electrocorticography and field potential intracerebral depth recordings. Both types of recording can be vulnerable, within seconds, to alterations in perfusion. These recordings can be a standard of functional activity because the encephalogram is highly susceptible to, and changes in specific fashion upon, alterations in blood pressure or perfusion. For example, brain reperfusion models that succeed at preserving single neuron and local electrical activity can be characterized by disruption or loss of the electroencephalographic signal. Thus, to assess preservation of cerebral activity under EPCC, neurophysiological recording conditions that provide for the measurement of pre-EPCC activity comparable to that typical of the awake human brain (as quantified by electrocorticography) can be used. Also used can be individualized pre- and post-EPCC recordings to evaluate neurophysiological impact.

In summary, a primary objective can be to preserve brain function under EPCC and/or to provide circulatory isolation from the majority of the rest of the body in a fully controllable fashion. Brain function can be inferred from the integrity of high-order network activity as reported by the quantification of broad-spatial scale electrical field potential recordings from the surfaces and depths of the brain.

Referring to FIGS. 14A-14C, the EPCC system is depicted showing the components discussed above. Custom-developed software can provide multiparameter physiological data visualization in a single interface and in real time via connection to a compactRIO (National Instruments). This software can provide an integration platform for multiple different devices from different manufacturers to be integrated into a single input/output system (e.g., a manufacturer-agnostic system) to implement the technology disclosed herein. For instance, in this manner, data including one or more of aortic pressure, Brachiocephalic pressure, carotid pressure, pump flow, brachiocephalic artery flow, pump motor velocity, or blood gas and electrolyte measurements can be synchronized. The motor part of EPCC can be an analog programmable centrifugal pump (e.g., BVP-ZX, Harvard Biosciences) that generates pulsatile waveforms. These waveforms can be modeled to replicate the native physiological pressure or flow recordings obtained from individual pig(s). The BVP-ZX can control perfusion via a magnetic impeller motor that prevents contact with the perfusate. A commercial pump head with reduced hemolytic action (e.g., BP-80, Medtronic) can be coupled to the pump. The measured native circulatory pressure waveforms can be converted to analog voltage waveforms and can be used to regulate centrifugal pump speed (RPM). This regulation can generate native-like pulsatile flow via custom-developed software using LabVIEW (National Instruments). The correlation between input voltage and RPM can be linear. The gains of the input can be manually adjusted to ensure adequate pressure and flow while maintaining native-like pulsatile pressure. Additional custom-developed software can provide the use of a series of .CSV files containing a broad range of waveforms that took into account native waveforms in order to modify pulsatile systolic and diastolic pressure and beats per minute (BPM) independently of one another or as desired. The custom software can provide linear, continuous flow.

The system can be complemented with a clinical oxygenator (e.g., Affinity NT, Medtronic), a continuous perivascular flowmeter (e.g., Transonic), a continuous fiber optic pressure meter catheter (e.g., 2 French and/or Fiso), a heat exchanger (e.g., LT Ecocool), a continuous blood gas monitor (e.g., CDI, Terumo) and/or medical grade perfusion tubing (e.g., ⅜ and/or Medtronic). Continuous circuit flow monitoring can use the inline flow probe located distal to the pump while flow measurement at the level of the brachiocephalic artery can use the perivascular flowmeter. The fiber optic pressure monitoring catheter can be inserted in-line to the flow of blood. Venous return from the superior vena cava return can be connected to the reservoir to maintain a closed perfusion circuit with the capability to add blood or electrolytes if needed.

Preliminary tests and adjustments of EPCC and its separate constituents can be conducted with either water containing polyethylene glycol-200 (MilliporeSigma) dissolved to approximate blood osmolality (300 mOsm 13) or pig blood. These studies can be performed on the mechanical circuit operating both in isolation and after connection to heparin-treated pig carcasses.

In some examples, a case study is performed in accordance with all animal research guidelines. This work can investigate numerous, variable aspects, with observations made in each carcass informing the approach to the next one in cumulative fashion. Second, additional preliminary studies can be conducted, also in cumulatively modified fashion, in 4 anesthetized juvenile pigs of 4-5 months of age weighing 20-25 Kg. This weight can be chosen because of progressive limitation to surgical intracranial accessibility with age. These pigs can be used to further develop isolated aspects of surgery, angiography or EPCC. Third, 6 pigs weighing 30-60 Kg used in unrelated terminal studies can be exsanguinated under general anesthesia and the heterologous blood stored in standard clinical citrate-phosphate-dextrose bags and stored at 4° C. for use in EPCC with other pigs within 2 days. The blood type of donors and recipients (i.e., those to be subject to EPCC) can be undetermined. Lastly, the results reported below can be obtained from two further anesthetized female pigs, hereafter referred to as subjects 1 and 2. Of note, no significant differences in somatic physiological or neurophysiological parameters measured in males and females can be found. One of these pigs (subject 1) can be used to illustrate vascular disconnection at the aorta and the other (subject 2) at the brachiocephalic artery. The method can preserve native circulation and brain activity, which can be assessed from the analysis of the recordings obtained over a sustained period of time (hours) from each of these two animals, rather than statistically from a group of animals.

In some instances, a monitoring, anesthesia, and/or intravenous fluids procedure can be performed. About 30 min prior to surgery, subjects 1 and 2 can be sedated with an intramuscular injection of tiletamine and zolazepam (4-8 mg/kg of each, in equal amount), atropine (0.04 mg/kg) and buprenorphine (0.05 mg/kg). They can then be administered inhaled isoflurane, except during neurophysiological recording as noted below, and oxygen (2 L/min). These gases can be applied first via snout mask and immediately afterwards via endotracheal intubation with mechanical ventilatory support. General anesthesia can be maintained throughout the rest of the life of the animals, including euthanasia. This included isoflurane at 1-2% v/v with air during non-surgical activities and higher concentrations (up to 5%) during the performance of incisional or manipulative surgery. The isoflurane can be replaced with intravenous anesthesia including ketamine (10-20 mg/Kg/hr), xylazine and guaifenesin using a dose ratio of 1:1:500 during the recording of neurophysiological activity under native and EPCC conditions. The respiratory rate can be assisted as needed with a standard animal respirator to maintain 15-20 breaths per minute. Intravenous and intra-arterial catheter (auricular and femoral) and esophageal electrocardiogram (EKG) probe and rectal temperature probe insertion can be also performed. Heart rate, arterial blood pressure, respiratory frequency, pulse oximetry and capnography can be thus monitored to document stability within standard veterinary ranges. An intravenous saline infusion can be maintained at 50 mL/h. During some of the preliminary studies and in subjects 1 and 2, 450 ml of heterologous blood can be infused just prior to large artery cannulation. This had no appreciable effect on native blood pressure.

In some instances, a cerebral exposure and recording procedure can be performed. For instance, each anesthetized pig can be first maintained prone on a padded operating table. A strap secured the torso to the table to prevent movement during cranial surgery. The body of the animal can be covered with a heated air blanket (e.g., Bair hugger, 3M) and the head can be surgically draped to allow cranial exposure. Anteroposterior brain length can be 5.9-6.2 cm for all the pigs weighing 20-25 Kg. Thus, the cranium can be partially excised as previously, taking into account both this dimension and the provision of electrode coverage over an ample surface of the cerebral convexity. The dura can then be opened to allow the apposition of two linear electrocorticography strips to the cerebral surface, each containing 4 electrodes separated by 1 cm, and the near-vertical insertion of two depth electrode linear arrays (Ad-tech), each containing 8 platinum cylindrical recording sites separated by 0.5 cm. These arrays traversed the entire brain in the craniocaudal direction on either side of the midline at the level of the coronal suture. In this configuration, they recorded activity spanning from the immediately subcortical white matter to the hippocampus. Alternatively, in some preliminary study cases the electrodes can be placed in the same configuration using 4 burr holes. A Neurovent-PTO 2L brain probe (Raumedic) can be inserted into the left frontal lobe at a depth of 1.5 cm for temperature, intracranial pressure, and tissue oxygen saturation monitoring. The scalp can then be sutured closed in fluid-tight fashion. The positions of the electrodes and brain probe can be documented radiographically aided by a radiopaque ruler. In some preparative cases, subdural radiopaque contrast fluid can be also injected to delineate the brain configuration relative to electrode placement. Estimated blood loss for these procedures can be 5 mL. Neurophysiological recordings can be conducted inside a custom designed Faraday cage using a clinical 32-channel amplifier (Neurofax EEG-1200, Nihon Kohden, Japan). The recording sampling rate can be 1000 Hz and a band-pass filter of 0.5 to 300 Hz can be applied. All other aspects of these procedures can be as described 12.

In some examples, a neurophysiological data analysis procedure can be performed. For instance, data from the recording electrodes can be analyzed. This included oscillation frequencies in the range of delta, theta, alpha, beta and gamma range. Welch's method for fast Fourier transform can be utilized to calculate power for each frequency during 5-min consecutive epochs. To measure temporal changes in frequency spectra, consecutive 10-min epoch depth activity from both pigs can be selected and absolute power can be calculated as described 12. Parietal strip electrodes can be selected for the calculation of power spectral density (PSD) shown below.

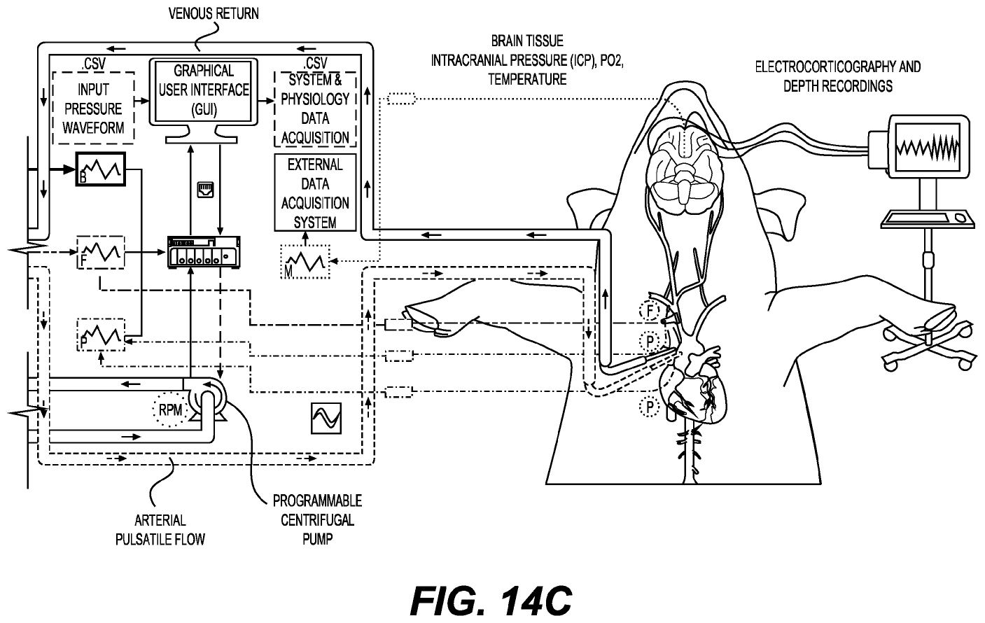

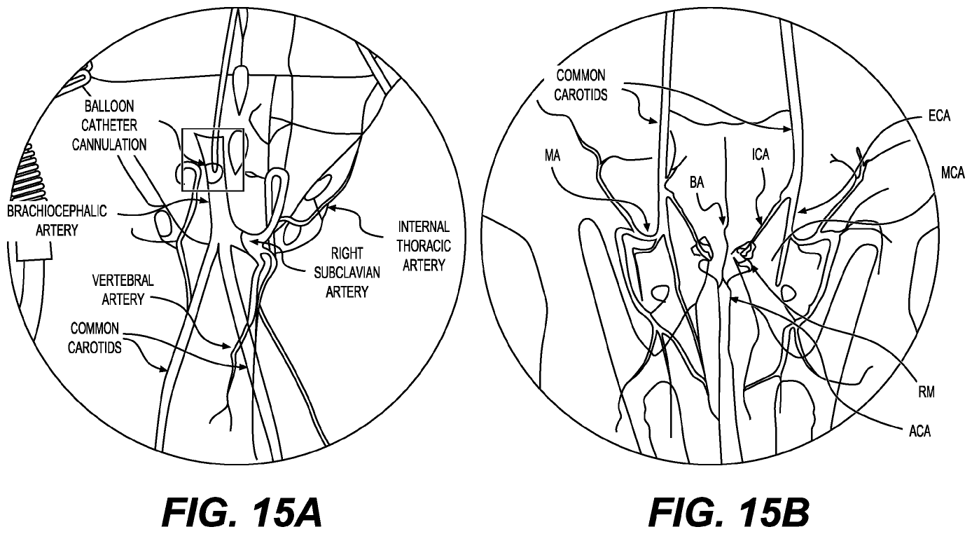

FIG. 15 illustrates an angiography of preliminary studies in 3 pigs which can be used to define the relevant vascular anatomy from the point of cannulation to the small arteries of the brain. The images depicted in FIG. 15 can be obtained at the end of an EPCC procedure. Radiographic contrast agent (e.g., Omnipaque, 2% diluted in saline, GE Healthcare) can be used in conjunction with an Elite CFD radiographic C-arm (e.g., GE Healthcare). At the conclusion of EPCC, a Foley catheter (e.g., Bard Medical) can be placed inside the brachiocephalic artery and the balloon can be inflated with air to fill the vessel. An arterial line can then be used to inject the contrast agent mixture followed by axial radiography.

Furthermore, FIG. 15 depicts vascular anatomy of chest and head, including a thoracic angiogram obtained via brachiocephalic cannulation (A) and extension of the radiographic field to the head (B) to illustrate the arteries derived from the carotids. The usual craniocaudal orientation is reversed in these radiographs, with the snout pointing toward the inferior part of the images. MA: mandibular artery; BA: basilar artery; ICA: internal carotid artery; ECA: external carotid; MCA: middle cerebral artery; ACA: anterior cerebral artery; RM: rete mirabile.

In some examples, the disclosed techniques can include a carotid artery recordings procedure. For instances, carotid artery exposure and cardiovascular surgery can be followed by supine inversion and positional securing of the pigs as above. The snout can be maintained at heart level using a cushioned support. Of note, while the human brachiocephalic, left common carotid, and left subclavian arteries emerge separately from the ascending aorta, pigs possess only two branches. The brachiocephalic artery gives rise to not only the right subclavian and right common carotid arteries, but the left common carotid artery as well 14. Thus, from a cerebral perfusion perspective, access to the brachiocephalic artery can ensure viability.

In some instance, the right common carotid artery can be dissected using electrocautery. Following exposure of 7-9 cm of the artery, the fiber optic catheter can be transiently threaded through a 22 G arterial puncture cannula and left in place 1-2 cm deep, with the tip pointing towards the brain, thus allowing the continuous measurement of arterial pressure before and after EPCC. Data acquisition can subsequently be initiated through interfacing the corresponding signal conditioning circuit with the LabVIEW controlled Compact-RIO 9045 for direct visual analysis and data storage. As such carotid native recordings can be used in subsequent comparisons with EPCC recordings. Proprietary software can be used concurrently with the main pressure data acquisition system as a backup.

In some examples, a native thoracic artery recordings procedure can be performed. For instance, simultaneously with or following carotid artery access, the thoracic cavity can be opened. Electrocautery can be used to expose the sternum and a power bone saw (e.g., Stryker) applied to the midline, thus dividing apart the rib cage. Following meticulous exposure of the large arterial and venous vessels from the heart to the thoracic inlet, a second 2 French fiber optic pressure catheter can be inserted into either the ascending aorta (subject 1) or the brachiocephalic artery (subject 2) for continuous native pressure measurements. The perivascular Doppler flowprobe can be fitted to encircle the brachiocephalic artery and immersed in a small amount of surgilube ultrasound gel (e.g., HR Pharmaceuticals). Estimated blood loss for these procedures can be 10 mL. Continuous native pressure and flow recordings (e.g., when applicable) can be visually displayed and stored through the Compact-RIO 9045 interface. Since circulatory stability can be documented in preliminary studies lasting over 4 hours, both probes can be removed following 5-10 min of data collection. A single representative native pressure waveform and the pulsation frequency can then be used as input to the pulsatile pump.

In some examples, the systems disclosed herein can perform a EPCC circuit priming procedure. For instance, the circuit can be primed separately from but simultaneously with the performance of vascular access surgery to minimize blood loss. Prior to the addition of heterologous blood, the Affinity NT reservoir can be primed with 1000 cc of 0.9% Sodium Chloride solution. 5,000 U of heparin can be added. Pump-induced positive pressure can establish flow from the reservoir to the heat exchanger/oxygenator and then to the rest of the vascular system upon connection. Priming can ensure proper fluid-solid interfacing with the tubing devoid of air bubbles. Just prior to EPCC, 600 mL of heterologous blood can be deposited into the venous reservoir. The blood can be filtered and continuously recirculated in a closed loop fashion prior to initiation of EPCC to remove air bubbles that may develop during reservoir decantation. Following circuit operation for up to 30 min prior to EPCC, the passing of blood through the heat exchanger/oxygenator can provide an initial and stable temperature of 37.2° C. and a fractional oxygen percentage of 80% can be maintained at 1.5 liters per minute of oxygen flow across the oxygenator.

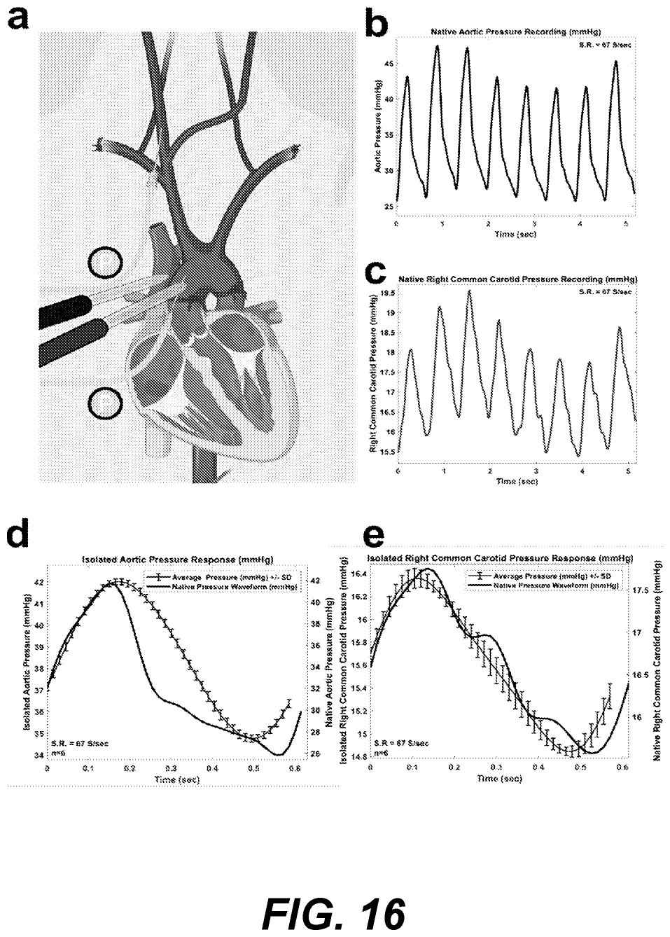

FIG. 16 illustrates one or more methods including an aortic isolation of subject 1. As depicted in FIG. 16, EPCC pressure can be shown relative to native perfusion pressure following aortic isolation (subject 1). FIG. 16 depicts (a) a diagram of vascular structure and aortic isolation with red indicating arteries and arterial cannula; blue indicating veins and venous cannula; and P (yellow) indicating pressure measurement locations; (b) native aortic pressure; (c) native right common carotid pressure; (d-e) comparative waveforms of 6 averaged recordings under EPCC and native waveforms used as input for the aortic pressure and for the right common carotid pressure, respectively. All recordings can be sampled (sampling rate: S.R.) at 67 samples per s.

For instance, FIG. 16 illustrates the connection of the aorta following its isolation. 300-400 U/kg of heparin can be intravenously infused. Incisions can be directly made immediately proximal to the placement of the pressure catheter for access with an 8 French fenestrated arterial cannula (Medtronic), which can be secured by suture immediately distal to the aortic valve and coronary arteries. This can prevent retrograde flow, thus ensuring devitalization of the heart. A venous straight cannula (e.g., Medtronic) can be placed immediately superior to the right atrium in the superior vena cava and can be secured through suture. Flow returning from the inferior vena cava and superior vena cava into the right atrium can be blocked by sutures. Further vascular isolation can employ a suture placed immediately past the branching of the left subclavian off the ascending aorta. This can eliminate perfusion to the descending aorta. Lastly, to prevent flow to the subclavian artery, a clamp can be placed immediately distal to the vertebral artery bifurcation. The 2 French thoracic pressure catheter can then be reintroduced to initiate isolated aortic recordings. Estimated blood loss for this and the following procedure can be 50 mL. The connections thus created between arterial and venous cannulas and the cardiopulmonary bypass tubing can provide for full EPCC operations.

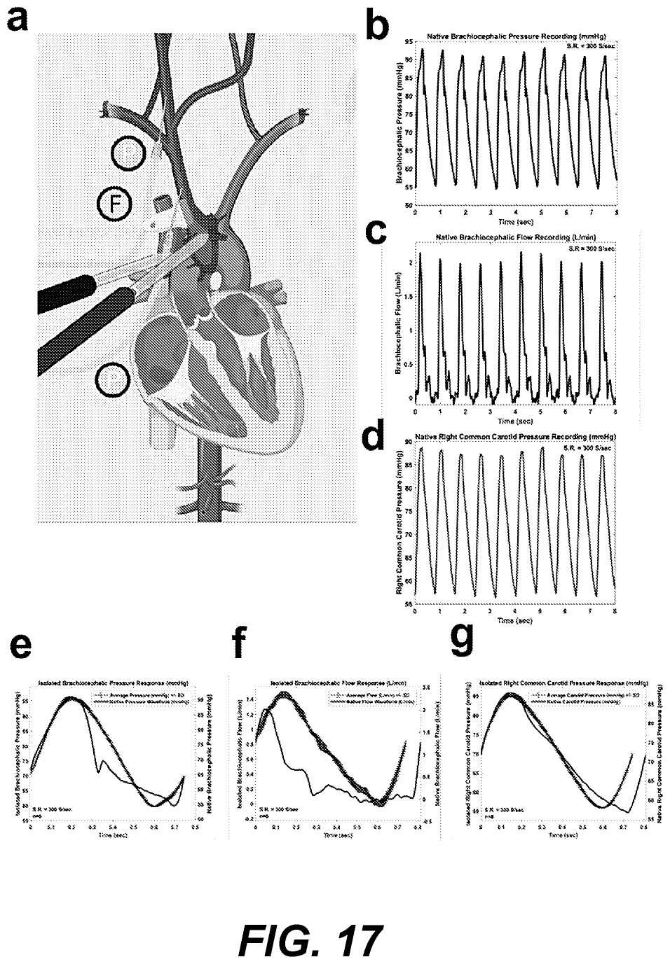

FIG. 17 illustrates an example brachiocephalic artery isolation procedure (subject 2). For instance, FIG. 17 depicts EPCC pressure relative to native perfusion pressure following brachiocephalic aortic isolation (subject 2). Accordingly, FIG. 17 shows (a) vascular structure and brachiocephalic isolation with red indicating arteries and arterial cannula; blue indicating veins and venous cannula; and P (yellow) indicating pressure measurement locations; and cyan indicating Perivascular Flow Measurement (F) (b) native brachiocephalic pressure (c) native Doppler-measured brachiocephalic flow; (d) native right common carotid pressure recordings; and/or (e-g) Comparative waveform analysis of six average recordings under EPCC and sampled native waveforms used as input for the brachiocephalic pressure, brachiocephalic flow and right common carotid pressure, respectively. All recordings can be sampled (sampling rate: S.R.) at 300 samples per sec.

In some instances, FIG. 17 shows the connection established after brachiocephalic isolation. The procedure can be similar to the aortic isolation procedure except for the placement of the arterial cannula, pressure catheter, incorporation of the perivascular flow probe and/or sutures. As with aortic isolation, incisions can be directly made immediately proximal to the placement of the measurement probes for insertion of an 8 French fenestrated arterial cannula. Unlike aortic isolation, immediately distally to the cannula, the perivascular flow probe is placed. The cannula can be secured through suture immediately proximal to a brachiocephalic branch. A venous straight cannula can be placed immediately superior to the right atrium in the superior vena cava and sutured in place. The apex of the ascending aortic arch can be sutured to prevent flow to the left subclavian artery and lower extremities, which can be different than the aortic isolation. Lastly, to prevent flow to the subclavian artery, a clamp can be placed immediately distal to the vertebral arterial bifurcation. The suture prior to the left subclavian branch can provide interruption of flow to the left vertebral artery. Whereas the right and left vertebral arteries can anastomose into the basilar artery, the left cerebellar hemisphere may experience altered perfusion, although circle of Willis anastomoses may compensate for this phenomenon. The 2 French pressure catheter can be placed immediately proximal to the flow probe at the base of the brachiocephalic branch.

In some examples, the systems disclosed herein can perform an EPCC initiation and data acquisition procedure. For instance, the tubing output from the flow probe can be connected to the arterial cannula inserted in the subjects. To avoid air bubbles, saline can be poured into the connecting ends. Using the same or a similar method as for arterial cannulation, the venous tubing can be connected to the cannula inserted into the superior vena cava, thereby closing the circuit. The pump can then be initiated on continuous flow for up to 3 min to ensure stability and to allow for circuit examination before switching to pulsatile flow. As noted above, the native pressure waveform and BPM can be used for input into the custom LabVIEW program to initiate EPCC. The carotid pressure, aortic or brachiocephalic pressure, flow, and pump motor performance can be collected through the LabVIEW interface and stored as a .CSV file for subsequent analysis. Any overt blood loss can be measured and compensated via supplementation of heterologous blood using the venous reservoir. Maximum blood replenishment can be 3.6 L in the course of 4 hr.

In some instances, a maximum EPCC duration can be 5 hr or longer with appropriate amounts of blood. The following principal parameters can be measured. Subject 1: Carotid pressure (mm Hg), aortic pressure (mm Hg), and motor response (RPM), sampled at 67 Hz, and blood gas and electrolyte analysis at 1 sample/min. Subject 2: Carotid pressure (mmHg), aortic pressure (mmHg), brachiocephalic flow, and motor response (RPM), sampled at 300 Hz, and blood gas and electrolytes as above.

In some examples, an additional hematocrit, gas and/or electrolyte analyses procedure can be performed. For instance, to assess the consistency of continuous CDI blood monitoring, blood samples can be collected from the reservoir for analysis every 5 min. This can include portable clinical analyses (e.g., sodium, potassium, chloride, CO2, anion gap, ionized calcium, glucose, urea nitrogen, creatinine, hematocrit and hemoglobin; I-stat 1, Abbott) and/or separate conventional clinical laboratory analyses of complete blood count and/or blood chemistry obtained after EPCC.

In some scenarios, a termination procedure can be performed. For instance, euthanasia can be induced at the conclusion of each study under general anesthesia by the intravenous addition of pentobarbital at excess dose (120 mg) sufficient to produce asystole, cessation of spontaneous respiration and the development of fixed and dilated pupils with absent corneal reflexes. Necropsy can be performed to verify electrode placement and brain configuration and integrity.

In some examples, a histological examination procedure can be performed on the brain. For instance, upon euthanasia, the brains can be subject to measurement of cerebral cortex thickness and layer organization following standard formalin fixation and paraffin embedding. Coronal sections can be exposed to Nissl/luxol fast blue as well as Nissl/periodic acid Schiff stains. Cortical layers can be examined and compared with the layering of the human cortex. The sections can be photographed on a microscope, such as a Leica DM2000 microscope equipped with a Jenoptik Gryphax NAOS CMOS camera.

In some scenarios, the surgical approaches discussed herein can have the results discussed herein. For instance, cranial surgery can be uneventful in all preliminary study pigs and in subjects 1 and 2. One preliminarily studied pig may have harbored a 3 cm right cerebral cortex tumor and one can be subclinically epileptic (i.e., no seizures had been previously apparent) as noted by electrocorticography. In some instances, there can be no, or few, appreciable untoward effects associated with the use of heterologous blood. Consistently with the cardiac dysfunction propensity upon manipulation of some domestic pigs, one preliminary study pig may have experienced ventricular fibrillation upon sternotomy and was terminated. Another preliminary study pig may have experienced cardiac hypokinesis after vascular manipulation that recovered after 15 min of cardiac massage and transfusion of 900 mL of heterologous blood.

In some examples, the systems disclosed herein can perform one or more perfusate composition stability processes. For instance, one preliminarily studied pig received 100 mEq of sodium bicarbonate into the blood reservoir to rectify a clinically significant reduction in blood pH (from 7.2; base deficit-5 mmol/L). All the blood samples analyzed in a clinical veterinary laboratory for subjects 1 and 2 yielded values within acceptable clinical veterinary ranges of variation relative to native samples.

Tables 1 and 2 below indicate these values. Table 1 shows the standard analysis of blood chemistry under EPCC. The blood analysis for Table 1 was performed using four samples obtained hourly under native (immediately pre-EPCC) and during EPCC conditions. Table 2 shows the cellular blood composition, with complete blood counts obtained during EPCC, to show a comparison with native values. For Table 2, four samples were obtained hourly for two of the subjects and every 1.5 hours for a third subject, and averaged. Standard hematological abbreviations are used.

TABLE 1

EPCC

| Analyte | Native | Mean | SD | Units |
|---|---|---|---|---|
| Total protein | 5.1 | 4.7 | 0.6 | g/dL |
| Albumin (A) | 2.1 | 2 | 0.3 | g/dL |
| Globulin (G) | 3 | 2.7 | 0.3 | g/dL |
| A/G ratio | 0.7 | 0.75 | 0.06 | |
| AST (SGOT) | 31 | 36.6 | 3.7 | IU/L |
| ALT (SGPT) | 50 | 46.8 | 6.3 | IU/L |
| Alkaline phosphatase | 90 | 76.8 | 9.0 | IU/L |
| Creatinine | 1.3 | 1.5 | 0.1 | mg/dL |
| Phosphorus | 9 | 10.8 | 2.1 | mg/dL |
| Glucose | 67 | 66.5 | 8.4 | mg/dL |
| Calcium | 9 | 8.3 | 0.5 | mg/dL |
| Sodium | 142 | 146.5 | 3.9 | mEq/L |
| Potassium | 3.9 | 4.7 | 0.7 | mEq/L |
| Chloride | 97 | 90 | 3.4 | mEq/L |
| Cholesterol | 69 | 59.5 | 8.7 | mEq/L |

TABLE 2

EPCC

| Count or index | Native | Mean | SD | Units |
|---|---|---|---|---|
| WBC | 13.3 | 9.3 | 1.9 | 10³/μL |
| RBC | 5.5 | 5.5 | 0.8 | 10³/μL |
| HGB | 8.6 | 8.7 | 1.2 | g/dL |
| HCT | 26 | 27.4 | 4.3 | % |
| MCV | 48 | 50.1 | 4.4 | fL |
| MCH | 15.8 | 16.1 | 0.6 | pg |
| MCHC | 33 | 32.4 | 1.9 | g/dL |
| Platelets | 101 | 83.5 | 69.6 | 10³/μL |
| Neutrophils | 8512 | 4077 | 844 | /μL |
| Lymphocytes | 3990 | 4885 | 1530 | /μL |
| Monocytes | 266 | 151 | 119 | /μL |
| Eosinophils | 532 | 222 | 138 | /μL |

Serial blood samples studied at the bedside (as shown in Table 3 below) also illustrated negligible or clinical acceptable variations in blood pH, CO2, O2, sodium, potassium, glucose among other analytes. This also suggests that hemolysis can be negligible and that the blood supplemented into the reservoir can be sufficient to maintain the metabolic homeostasis of the perfused tissue. The blood analyses depicted in Table 3 were performed with an I-stat analyzer for a subject under native (e.g., immediately pre-EPCC) and during EPCC conditions, with three samples obtained every 1.5 hours.

TABLE 3

Perfusate

| Parameter | Native | Mean | SD |
|---|---|---|---|
| pH | 7.64 | 7.47 | 0.07 |
| PCO² (mmHg) | 26.2 | 36.1 | 2.1 |
| PO₂ (mmHg) | 451 | 365 | 91 |
| HCO₃ (mmol/L) | 28.1 | 26.4 | 2.8 |
| TCO₂ (mmol/L) | 29 | 27.7 | 2.5 |
| Saturation O₂ (%) | 100 | 100 | 0 |
| Na (mmol/L) | 136 | 129 | 3.5 |
| K (mmol/L) | 3.4 | 4.6 | 0.2 |
| Ionized Ca (mmol/L) | 0.54 | 0.34 | 0.01 |
| Glucose (mg/dL) | 291 | 340 | 55 |
| Hematocrit (%) | 19 | 20.3 | 6.5 |
| Hemoglobin (g/dL) | 6.5 | 5.3 | 4.8 |

In some scenarios, pressure and flow measurements at the carotid arteries can be performed. For instance, native aortic pressure recordings measured from subject 1 for the purpose of physiological replication under isolated conditions can be sampled at 67 Hz. An 8-iteration (e.g., 8-beat) recording is depicted at FIG. 16(b). Under native conditions, subject 1 can exhibit an average aortic systolic pressure of 43.9+/−2.26 mmHg and an average aortic diastolic pressure of 26.434+/−0.686 mmHg. Average native mean arterial pressure (MAP) averaged out at 32.26+/−1.262 mmHg, with a heart rate of between 98 and 99 BPM. A representative aortic waveform with a systolic and diastolic pressure of 41.72 and 25.81 mmHg, respectively, can be used as an input waveform for pump control. The mean arterial pressure (MAP) can be calculated at 31.12 mmHg. The response to the pump input measured in the aorta is depicted at FIG. 16(d) at the specified heart rate of 98.7 BPM. Six consecutive waveforms can be averaged for further analysis. This can indicate that the aortic pressure under EPCC can be, on average, 42.016+/−0.14 mmHg for the systolic and 34.76+/−0.16 mmHg for the diastolic level, yielding a MAP of 37.18 mmHg. RPM analysis can indicate that the pump consistently provided cycles between 1761.456 and 1352.966 revolutions per second to generate these systolic and diastolic pressures.

In some examples, carotid pressure recordings can also be sampled at the same rate. FIG. 16(c) represents a corresponding 8-iteration of native carotid pressure recordings in response to the most significant aortic pressure fluctuations noted. Significant dampening of the carotid pressure waveform can be observed. Corresponding native carotid recordings had an average systolic pressure of 18.49+/−0.66 mmHg while average diastolic pressures were 15.9+/−0.40 mmHg. This resulted in an average carotid MAP of 16.76+/−0.47 mmHg. The native systolic and diastolic carotid pressure corresponding to the aforementioned aortic input pressure is observed to have a systolic pressure of 17.75 mmHg and a diastolic pressure of 15.57 mmHg, respectively, providing a MAP of 16.3 mmHg. The carotid pressure response under EPCC in comparison with the native pressure waveform is illustrated in FIG. 16(e). Further analysis indicated that the EPCC pressure response in the carotid artery resulted in an average systolic/diastolic pressure ratio of 16.36+/−0.09 mmHg to 14.84+/−0.05 mmHg, respectively, thereby resulting in a MAP of 15.35 mmHg.

In some scenarios, native and EPCC brachiocephalic and/or carotid pressure recordings obtained from subject 2 can be recorded at a sampling rate of 300 per second. A subset of native representative physiological recordings (10 iterations) taken for EPCC replication are depicted in FIG. 4B-D. Brachiocephalic pressure, measured near the origin of the artery, consisted of an average systolic pressure of 91.73+/−0.94 mmHg and an average diastolic pressure of 54.86+/−0.47 mmHg. This results in a calculated average MAP of 67.15+/−0.61 mmHg. Average native heart rate can be approximately 75 BPM. Native brachiocephalic flow can be 6.2242 mL per beat. Right common carotid native recordings exhibited undiminished tonicity, providing a uniform average systolic to diastolic ratio of 87.6+/−0.69 mmHg to 56.84+/−0.36 mmHg mmHg, yielding an average MAP of 67.10+/−0.44 mmHg, similarly to the values obtained in the brachiocephalic.

FIG. 17(e)-(g) represents a waveform comparative analysis between native and EPCC pressure and flow to illustrate mechanical fidelity. As noted for native measurements, EPCC using a preset native waveform (taken from subject 1) can be amplified to provide subjective native systolic/diastolic ratios subjected to a heart rate of 80 BPM. The results indicate that, under EPCC using an input pressure waveform with a systolic/diastolic ratio of 90.82 mmHg/54.49 mmHg and a MAP of 66.6 mmHg, which were selected using the extrema from the native brachiocephalic dataset, the output brachiocephalic pressure recordings (evaluated using 6 averaged waveforms) can be averaged to provide a systolic to diastolic ratio of 95.8+/−0.63 mmHg to 59.68+/−0.13 mmHg with an MAP of 71.7 mmHg. In comparison to a corresponding native carotid pressure measurement of 86.93/56.75 mmHg, yielding a MAP of 66.8 mmHg, the EPCC carotid pressure can be, on average, 85.0+/−0.7 mmHg/55.9+/−0.2 mmHg with a MAP of 65.6 mmHg. Flow analysis using six averaged pressure waveforms can yield 9.011 mL per beat. The maintenance of the desired brachiocephalic and common carotid pressures can be provided by a consistent RPM systolic-diastolic ratio of 2556.3/1461.06 RPM.

While the significant pressure waveform similarities noted between native and EPCC conditions can indicate that both aortic and brachiocephalic flow approaches provide for the faithful replication of native waveforms, the results indicate that control of flow through the brachiocephalic approach leads to limited pressure dampening between the arterial cannulation site and the common carotid artery. Additionally, the ability to maintain almost identical systolic/diastolic pressure to native conditions under EPCC suggests improvements to the brachiocephalic approach.

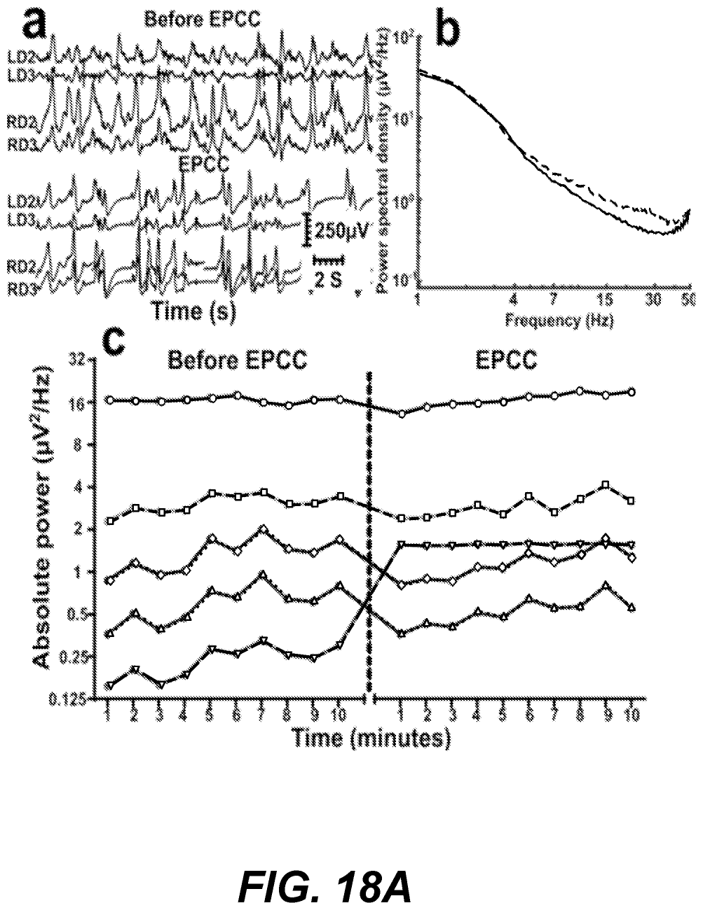

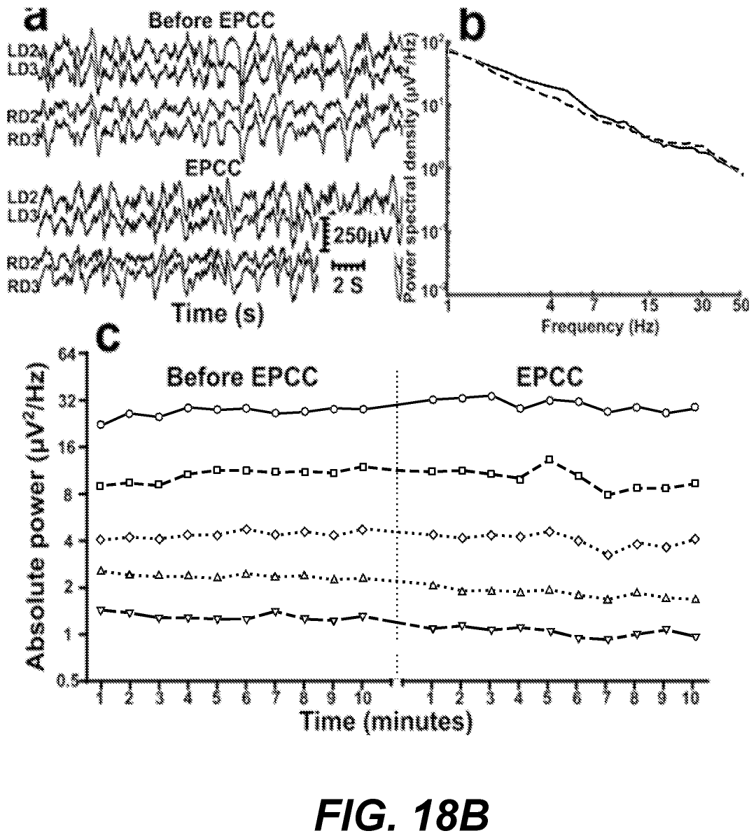

FIGS. 18A and 18B depict cerebral activity under EPCC which can be measured and/or recorded using the techniques disclosed herein. EPCC can be compatible with the sustained preservation, without interruption, of electrical activity both in subject 1 (FIG. 18A) and subject 2 (FIG. 18B). Both craniotomy and burr hole approaches can provide virtually indistinguishable recordings. The latter approach can provide the preservation of a greater intracranial pressure by about 5 mmHg, which may be relevant to maintain cerebral perfusion pressure commensurate with the likely native pressure. Of note, characteristic electrocorticography and depth recordings from the pig brain can be previously illustrated using craniotomy and other means essentially identical to the present study and this can provide comparison of those recordings with human awake electrocorticography. The results from subjects 1 and 2 and from three additional preliminarily studied pigs can be virtually indistinguishable from these previous recordings.

FIG. 18A illustrates a comparison of depth neurophysiological activity recordings before and after aortic EPCC (for subject 1). For instance, FIG. 18A shows (a) depth activity from left (L) and right (R) depth (D) electrodes corresponding to subcortical brain regions spanning from the subcortical white matter (LD2 and RD2) to the dorsal striatum (LD3 and RD3) before and following EPCC; (b) absolute power spectral density of native (blue spectra), post-EPCC (black dotted spectra) LD2 depth recordings in subject 1; (c) absolute power spectra of delta (red), theta (green), alpha (black), beta (blue), and gamma (magenta) activity as defined in standard electroencephalography and measured in LD2 using 10-minute epochs under native perfusion and under EPCC.

FIG. 18B illustrates a comparison of depth neurophysiological activity recordings before and after brachiocephalic EPCC (for subject 2) before and after EPCC. For instance, FIG. 18B shows (a) depth activity from left (L) and right (R) depth (D) electrodes corresponding to subcortical brain regions spanning from the subcortical white matter (LD2 and RD2) to the dorsal striatum (LD3 and RD3) before and following EPCC; (b) absolute power spectral density of native (blue spectra), post-EPCC (black dotted spectra) LD2 depth recordings in subject 1; (c) absolute power spectra of delta (red), theta (green), alpha (black), beta (blue), and gamma (magenta) activity as defined in standard electroencephalography and measured in LD2 using 10-minute epochs under native perfusion and under EPCC.

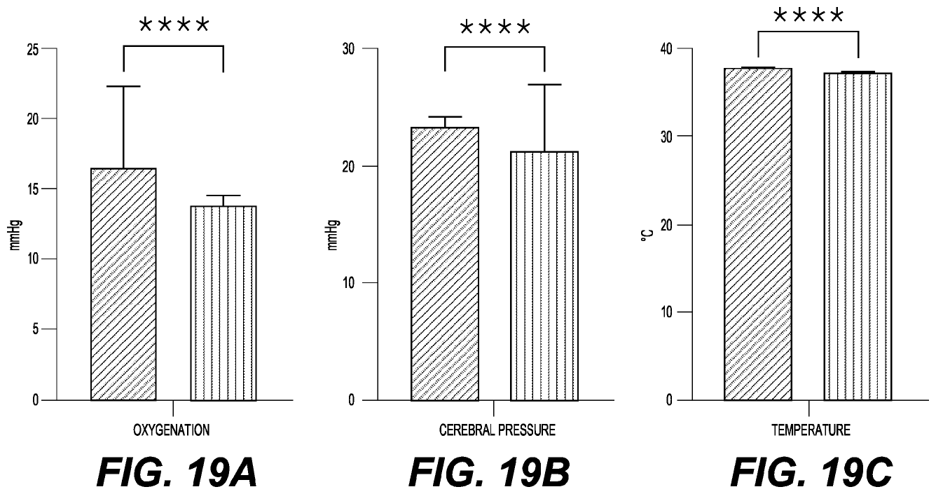

FIG. 19 depicts bar graphs showing that the native intracerebral pressure and temperature can be also maintained by the systems disclosed herein. Tissue oxygenation, however, can be greater than in native conditions, likely because of oxygen administration. FIG. 19 depicts physical characteristics of cerebral tissue after EPC, brain probe measurements of frontal lobe cerebral oxygenation (mmHg of oxygen), barometric pressure (mmHg) and temperature (° C.) in subject 2. Black bars represent average and SD of measurements obtained in the native, pre-EPCC state for 10 minutes with a sampling rate of 1 Hz. Gray bars indicate averaged values of 10 minutes epochs under EPCC, measured over 5 hr.

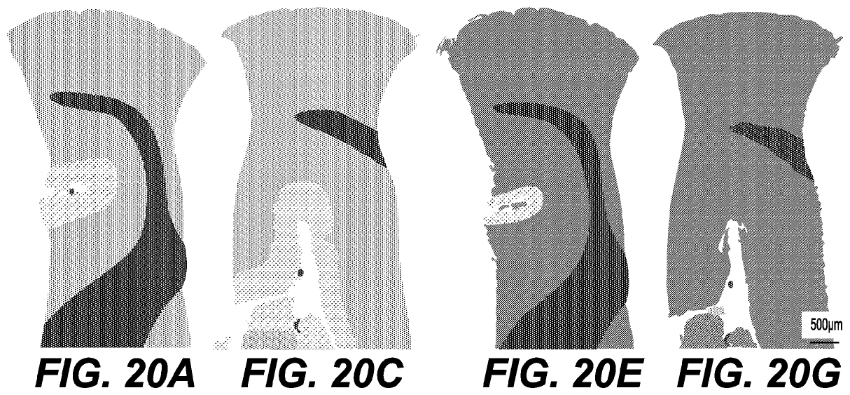

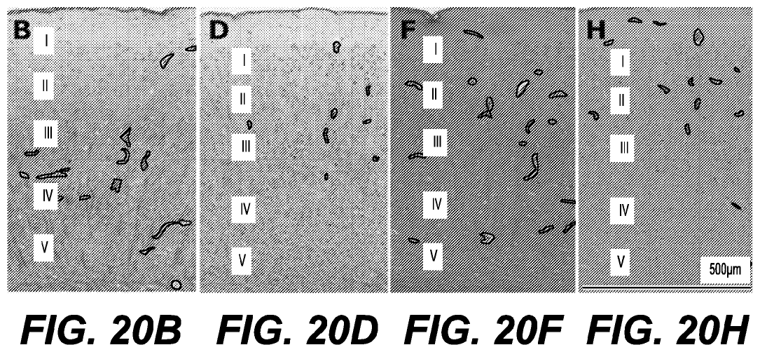

FIGS. 20A-20H depicts a microscopic structure of the brain cortex which can result from the systems disclosed herein. For instance, sections obtained throughout several brain regions can document preservation after EPCC relative to untreated pigs. Cortical cell structure and layer distribution (e.g., exemplified in FIG. 20 for the somatosensory cortex, with additional normative examples and human comparison in 12) can be unaltered by optic microscopy using both Nissl/luxol fast blue and Nissl/periodic acid Schiff staining examined under 10 and 20× magnification. Specifically, there can be an absence of abnormal neurons and white matter abnormalities.

In some instances, the preservation of cerebral activity under EPCC can be extended for the duration of each subject study (e.g., 5 hours). Except for excess brain tissue oxygenation upon oxygen supplementation, or intracranial pressure changes when craniotomy is used, this can be associated with near-native levels of cerebral physiological parameters such as intracranial pressure, tissue oxygen saturation and temperature.

Furthermore, measurements can be taken of the neurophysiological activity recordable by contact with the cerebral cortex and with deep structures particularly susceptible to ischemia. The division of this activity into standard spectral oscillatory frequencies and the selectivity and velocity of changes noted for some of these frequencies after ischemia can indicate that EPCC imposes no significant circulatory changes on the brain relative to native perfusion. In fact, because even profound neurophysiological changes during carotid endarterectomy can prove fully reversible, no cellular injury is expected from EPCC. Thus, the technology disclosed herein merits consideration for the investigation of cellular signaling and other mechanisms likely to be perturbed under linear flow conditions. Of note, in the pig, and in contrast with other animals, carotid artery flow can be closely correlated with cerebral blood flow, such that measurements at the carotid can be endowed with direct significance in terms of impact on cerebral blood flow.

In scenarios with a cerebral blood flow of approximately 90 mL/min per 100 g and a head (without the brain) blood flow of 10 mL/min per 100 g 17, an estimation can be made of an EPCC blood flow of 150 mL/min to the head (except the brain) and 54 mL/min to the brain. These estimates are subject to significant pig breed-specific head tissue composition (which primarily includes bone and muscle) variability. However, the relative tissue abundance of the head can be determined via several methods. Moreover, blood flow to these individual tissues can be estimated for the pig. Therefore, in some scenarios, after these calculations, it is possible to simplify metabolic tracer or pharmacological kinetic studies using EPCC. Further, the blood distribution volumes and metabolic activities of bone and resting head muscle are far below those of other organs such as liver and kidney, thus reducing the additional physiological complexities that curtail such studies.

In some instances, upon artificial perfusion, isolated pig brains can retain both cellular configuration and activity for several hours post-mortem. However, the electrocorticogram can remain isoelectric. The relative contribution to this isoelectricity of the perfusate composition and of the intrinsic suppression of field potentials after artificial perfusion can be non-quantified. However, it is likely that the electrocorticogram, which reflects the summation of a large ensemble of field potentials not made apparent by single-unit and other localized recordings, indicates cessation of synaptic critical for high-order network activity. This is implicit in the use of the loss of the electroencephalographic signal for the diagnosis of brain death.

In some examples, durations for the EPCC can be 5 hours although longer artificial perfusion times are feasible. Rectification of blood chemistry or hematocrit can be unnecessary in this study interval, likely because of heterologous blood replenishment of blood losses. Maintenance for prolonged times of infraphysiological cerebral perfusion pressure due to craniotomy may result in cerebral edema.

In some instances, ketamine anesthesia during the acquisition of the neurophysiological recording segments can be used for comparison with previously collected data. Ketamine can be beneficial given its reduced impact on brain activity, including a lack of depression of the cerebral oxygen metabolic rate. Rather, ketamine increases oxygen consumption slightly in association with increased metabolite supply. This may amount to a ˜15% increase in regional glucose metabolic rate (when studied at sub-anesthesia concentrations) but altered coupling or disequilibrium between cerebral blood flow and metabolism is unlikely.

While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an implementation in the present disclosure can be references to the same implementation or any implementation; and, such references mean at least one of the implementations.

Reference to “one implementation” or “an implementation” means that a particular feature, structure, or characteristic described in connection with the implementation is included in at least one implementation of the disclosure. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various implementations given in this specification.

Without intent to limit the scope of the disclosure, examples of instruments, apparatus, methods and their related results according to the implementations of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

1. A method for blood perfusion, the method comprising:
receiving, via a graphical user interface presented to a user, datapoints indicating a waveform;
receiving one or more parameters associated with blood perfusion;
generating an offset removed waveform based on the datapoints, the offset removed waveform having a physiological offset removed;
converting the offset removed waveform to a voltage waveform based on the one or more parameters; and
operating, via the voltage waveform, a pump to provide blood in a perfusion system.

2. The method of claim 1, wherein the waveform includes an irregular waveform.

3. The method of claim 1, further comprising providing a metabolite infusion to a subject of the perfusion system.

4. The method of claim 1, wherein the waveform includes a portion of a measurement associated with an isolated organ.

5. The method of claim 1, wherein the one or more parameters includes at least one of a beats per minute (BPM) parameter, a systolic pressure, flow, or voltage parameter, a diastolic pressure, flow, or voltage parameter, file size, or data acquisition rate, a waveform input, a file saving location, or data textual commenting.

6. The method of claim 1, wherein the pump includes a centrifugal pump.

7. The method of claim 1,
wherein,
the receiving of the one or more parameters includes receiving, via the graphical user interface, a BPM parameter, and
the datapoints are outputted for conversion to the voltage waveform based on the BPM parameter.

8. The method of claim 1,
wherein,
the receiving of the one or more parameters includes receiving, via the graphical user interface, at least one of a systolic pressure or flow parameter, a diastolic pressure or flow parameter, a systolic voltage, or a diastolic voltage,
the method includes amplifying the voltage waveform based on at least one of:
the systolic pressure or flow parameter and the diastolic pressure or flow parameter; or
the systolic voltage and the diastolic voltage, and
the operating of the pump includes providing the amplified voltage waveform to the pump.

9. The method of claim 1, further comprising:
receiving, from one or more sensors, at least one of a pressure measurement or a flow measurement from the perfusion system; and
operating the pump using feedback-based control responsive to the pressure measurement or the flow measurement.

10. The method of claim 1, wherein a rotation per minute (RPM) of the pump is varied based on the voltage waveform.

11. The method of claim 1, wherein the pump includes a magnetically coupled pump head.

12. The method of claim 1,
wherein,
the perfusion system includes a reservoir and an oxygen pressure mixer, and
the pump is coupled between the reservoir and the oxygen pressure mixer and configured to provide a blood flow from the reservoir to the oxygen pressure mixer.

13. The method of claim 12, wherein the perfusion system includes a heat exchanger configured to warm up the blood to be provided to the oxygen pressure mixer.

14. The method of claim 12, wherein blood from the oxygen pressure mixer is provided, via an isoflurane-controlled chamber, to the oxygen pressure mixer.

15. The method of claim 12, wherein blood from the oxygen pressure mixer is operable to flow to an aorta of a subject or an isolated organ of the subject.

16. The method of claim 12, wherein the perfusion system includes a shunt path coupled between a sensor and an arterial line coupled to an output of the oxygen pressure mixer.

17. The method of claim 12,
wherein,
the reservoir includes an input coupled to a venous line, and
the perfusion system includes a shunt path from the venous line to a sensor.

18. The method of claim 1, wherein the waveform comprises a pressure waveform, a flow waveform, or a physiological replicated voltage waveform.

19. A non-transitory computer-readable medium comprising at least one instruction for causing at least one of a computer or a processor to perform operations, the operations including:
receiving, via a graphical user interface presented to a user, datapoints indicating a waveform;
receiving one or more parameters associated with blood perfusion;
generating an offset removed waveform based on the datapoints, the offset removed waveform having a physiological offset removed;
converting the offset removed waveform to a voltage waveform based on the one or more parameters; and
operating, via the voltage waveform, a pump to provide blood in a perfusion system.

20. An apparatus comprising:
at least one processor; and
a memory including instructions that, when executed by the at least one processor, cause the apparatus to:
receive, via a graphical user interface presented to a user, datapoints indicating a waveform;
receive one or more parameters associated with blood perfusion;
generate an offset removed waveform based on the datapoints, the offset removed waveform having a physiological offset removed;
convert the offset removed waveform to a voltage waveform based on the one or more parameters; and
operate, via the voltage waveform, a pump to provide blood in a perfusion system.

21-22. (canceled)
